(19) United States
(12) Patent Application Publication
(10) Pub. No.: US 2012/0245051 A1
(43) Pub. Date: Sep. 27, 2012

(54) OBJECTIVE, QUANTITATIVE METHOD TO PREDICT HISTOLOGICAL SUBTYPE IN NON-SMALL CELL LUNG CANCER

(76) Inventors: David L. Rimm, Branford, CT (US); Valsamo Anagnostou, New Haven, CT (US)

(21) Appl. No.: 13/502,079

(22) PCT Filed: Oct. 13, 2010
(86) PCT No.: PCT/US10/52471

§ 371 Date: Jun. 14, 2012

Related U.S. Application Data
(60) Provisional application No. 61/278,965, filed on Oct. 13, 2009.

(51) Int. Cl. G01N 33/574; G01N 21/64; C40B 30/04; G01N 21/76

(52) U.S. Cl. 506/9; 435/7.92; 435/7.1; 435/7.23

(57) ABSTRACT

The invention provides a method for determining the histotype of a non-small cell lung cancer tumor which comprises: (a) determining the levels of expression of each of thyroid transcription factor-1, cytokeratin-5, cytokeratin-13 and epidermal growth factor receptor in a sample from the non-small cell lung cancer tumor; (b) calculating a score based on the levels of expression determined in step (a); and (c) comparing the score obtained in step (b) with a predetermined reference score associated with histotypes of non-small cell lung cancer; wherein the tumor is determined to be an adenocarcinoma if the score obtained in (b) is greater than the predetermined reference score and wherein the tumor is determined to be a squamous cell carcinoma if the score obtained in (b) is less than the predetermined reference score.

This application claims priority of U.S. Provisional Application No. 61/278,965, filed Oct. 13, 2010, the entire content of which is hereby incorporated by reference into this application.

Throughout this application, various publications are referenced by footnotes and/or parentheses. Full citations for these publications may be found at the end of the specification immediately preceding the claims. The disclosures of each of these publications is hereby incorporated by reference into this application in order to more fully describe the state of the art as known to those skilled therein as of the date of this application.

This invention was made with support under Grant No. 2009-0097 from the Connecticut Department of Health.

### FIELD OF THE INVENTION

This invention relates to the field of predicting histological subtypes in non-small cell lung cancer.

### BACKGROUND OF THE INVENTION

Clinical studies of existing and emerging therapeutics to treat non-small cell lung cancer (NSCLC) have shown differential therapeutic efficacy and side effect profile between the Adenocarcinoma (AC) and the Squamous subtype (SCC) of NSCLC. Because of the adverse activity of some of these agents in Squamous NSCLC patients there is a critical need to accurately identify the histotype of NSCLC patients prior to commencing therapy best suited for their particular tumor type. Currently there is no standardized assay that can adequately stratify patients for such treatments.

NSCLC is characterized by assessing morphology by histological analysis of the tumor biopsy. In addition a number of protein biomarkers such as Thyroid Transcription Factor-1 (TTF-1), Cytokeratin-5/6 (CK5/6), and Tumor Protein-63 (TP63) are used to sub-classify NSCLC histotype. These current methods are known to have poor reproducibility.

Other genes, microRNAs and proteins continue to be evaluated as potential biomarkers to improve on the accuracy and efficiency of differentiating the subtype of NSCLC. None of these emerging tests has demonstrated the ease of use, the accuracy, or the reproducibility that a test used in this clinical setting should demonstrate.

Described here is a quantitative, reproducible and easily applicable protein based test that accurately determines the sub-classification of a NSCLC tumor.

Expression of the following selected protein biomarkers was quantitated in formalin-fixed, paraffin-embedded (FFPE) tumor specimens using for example, AQUA technology, as previously described (Camp at al 2002 Nature Medicine 8(11)1323-1327, U.S. Pat. No. 7,219,016; Gustavson at al AQUA® technology and Molecular Pathology in Molecular Pathology in Drug Discovery and Development, Platero ed. John Wiley & Sons, Inc, Hoboken, N.J. 2009): Cytokeratin-5 (CK-5), Thyroid Transcription Factor-1 (TTF-1), Cytokeratin-13 (CK-13), Epidermal Growth Factor Receptor (EGFR), Phospho-p70-S6K, MENA, Cytokeratin-14, HER2, Cleaved Caspase-3 and Cytokeratin-17

These proteins were shown to be differentially expressed between the Adenocarcinoma subtype and the Squamous subtype of NSCLC. Based on the quantitation of these proteins, or a subset thereof, a method to determine the histotype of a NSCLC sample was developed. In particular, a protocol is described whereby the probability a NSCLC tissue sample is an adenocarcinoma versus a squamous cell carcinoma is calculated from the quantitative measurement of the expression of a subset of these proteins in the tissue sample. In one embodiment the subset of proteins includes: CK-13, EGFR, CK-5 and TTF-1. This method demonstrated a sensitivity of 97.2% and specificity of 94.8%. The method may be used in FFPE sections, fine-needle aspirates and other histological sample types. The method may be used to determine the likelihood a patient will benefit, or will be harmed by an intended therapeutic treatment. Treatments may include erlotinib, gefitinib, bevacizumab, sorafenib, docetaxel, gemcitabine, pemetrexed, cisplatin, alone or in combination with each other or other treatments. The method may determine a NSCLC is an adenocarcinoma whereby erlotinib, gefitinib, bevacizumab, sorafenib, sunitinib, docetaxel, gemcitabine, pemetrexed, cisplatin, alone or in combination with each other or other treatments may be beneficial treatments. The method may determine a NSCLC is a squamous cell carcinoma whereby bevacizumab, sorafenib, sunitinib, alone or in combination with each other or other treatments are not recommended treatments.

This biomarker based assay can also easily be applied to tumor material obtained by techniques less invasive than surgical biopsy such as bronchoscopic biopsies and fine-needle aspiration.

### SUMMARY OF THE INVENTION

The invention provides a method for determining the histotype of a non-small cell lung cancer tumor which comprises; (a) determining the levels of expression of each of thyroid transcription factor-1, cytokeratin-5, cytokeratin-13 and epidermal growth factor receptor in a sample from the non-small cell lung cancer tumor; (b) calculating a score based on the levels of expression determined in step (a); and (c) comparing the score obtained in step (b) with a predetermined reference score associated with histotypes of non-small cell lung cancer; wherein the tumor is determined to be an adenocarcinoma if the score obtained in (b) is greater than the predetermined reference score and wherein the tumor is determined to be a squamous cell carcinoma if the score obtained in (b) is less than the predetermined reference score.

The invention provides a method for determining whether a non-small cell lung cancer tumor is an adenocarcinoma which comprises (a) determining the levels of expression of each of thyroid transcription factor-1, cytokeratin-5, cytokeratin-13 and epidermal growth factor receptor in a sample from the non-small cell lung cancer tumor; (b) calculating a score based on the levels of expression determined in step (a); and (c) comparing the score obtained in step (b) with a predetermined reference score associated with histotypes of non-small cell lung cancer; wherein the tumor is determined to be an adenocarcinoma if the score obtained in (b) is greater than the predetermined reference score.

The invention provides a method for determining whether a non-small cell lung cancer is a squamous cell carcinoma which comprises (a) determining the levels of expression of each of thyroid transcription factor-1, cytokeratin-5, cytokeratin-13 and epidermal growth factor receptor in a sample from the non-small cell lung cancer tumor; (b) calculating a score based on the levels of expression determined in step (a); and (c) comparing the score obtained in step (b) with a predetermined reference score associated with histotypes of non-small cell lung cancer; wherein the tumor is determined to be an squamous cell carcinoma if the score obtained in (b) is less than the predetermined reference score.

The invention provides a method for selecting an appropriate therapeutic treatment or combination of therapeutic treatments for a patient afflicted with non-small cell lung cancer comprising (a) determining the levels of expression of each of thyroid transcription factor-1, cytokeratin-5, cytokeratin-13 and epidermal growth factor receptor in a sample of the non-small cell lung cancer tumor from the patient; (b) calculating a score based on the levels of expression determined in step (a); and (c) comparing the score obtained in step (b) with a predetermined reference score associated with histotypes of non-small cell lung cancer; (d)assigning a histotype to the non-small cell lung cancer wherein the tumor is determined to be an adenocarcinoma if the score obtained in (b) is greater than the predetermined reference score and wherein the tumor is determined to be a squamous cell carcinoma if the score obtained in (b) is less than the predetermined reference score; and (e) selecting a therapeutic treatment based on the histotype assigned in step (d).

### BRIEF DESCRIPTION OF THE FIGURES

FIG. 1. Antibody validation process. Antibodies were validated by AQUA and WB in known cell line positive controls as well as a series of cell lines variously expressing the proteins of interest. The dynamic range of CK13 (A), CK5 (B), EGFR (C) and TTF1 (D) expression by WB in cell lines controls was consistent with AQUA analysis. Representative immunofluorescence staining for A431 and HCC193 are shown; AQUA scores are displayed in insets.

FIG. 2. predictive value of the classifier in the training set. (A) Distribution of scores for specimens of the training set; SCCs (n=150, blue squares) have significantly lower scores compared to ACs (n=125, red squares). The dashed line at a score of 0.49 shows the optimal cut off point for an AC vs. SCC classification; that value had the maximum J index on the ROC curve. (B) ROC curve showing the sensitivity and specificity for various cut off values; the optimal cutpoint is circled. Sensitivity and specificity for cut off set at 0.49 were 96% and 93.3% respectively with an area under the curve of 0.975.

FIG. 3. Predictive value of the classifier in testing and validation cohorts. ROC curves for samples of the validation cohort 1(A) and 2 (8), using the same cut off point of 0.49 the 4-protein classifier yielded a sensitivity, specificity and AUC of 92.3%, 96.6% and 0.984 for validation set 1 and 96%, 96.7% and 0.989 for validation cohort 2. Specimen scores in ascending order are shown in inset.

FIG. 4. Classification of AS and LCC and giant cell tumors by the 4 protein assay. Application of the 4-protein classifier in AS (n=14, light blue squares) revealed that AS tumors could be classified as either AC or SCC (A); however our assay was unable to classify LCC and giant cell carcinomas that spanned the full spectrum between 0 and 1 (8).

FIG. 5. Schematic of the classifier development and validation. Consort diagram summarizing the main steps for development and validation of the 4-protein classifier: 24 proteins were measured in order to identify biomarkers preferentially expressed in ACs or SCCs; 8 proteins were further selected for analysis in a logistic regression model following a stepwise selection process. The final classifier was validated using 1000 bootstrap samples and a score was calculated for each specimen based on the formula score=1/(1+e−(9.679−0.899*CK13+0.711*TTF1−0.687*CK5−0.427*EGFR)). The classifier was then validated in 2 independent NSCLC cohorts.

FIG. 6. Assay reproducibility. Linear regression between AQUA scores of the specimens on serial cuts of the NSCLC control array provided a standard curve in order to normalize for minimal run to run variability; Pearson's R=0.91, p<0.0001 for CK13 (A), R=0.97, p<0.0001 for CK5 (B), R=0.91, p<0.0001 for EGFR (C) and R=0.95, p<0.0001 for TTF1 (D) runs.

FIG. 7. Quantitative immunofluorescence for CK5, CK13, TTF1 and EGFR. Tumor histospots corresponding to an AC (top row) and a SCC (bottom row) tumor: the cytokeratin-Cy3 image was used to identify the tumor component; the epithelial tumor “mask” is displayed in inset. The non nuclear (green) and nuclear (blue) compartments are defined by the cytokeratin and the DAPI signal respectively. CK13, EGFR, CK5 and TTF1-Cy5 images show representative expression patterns; auto-contrast of the EGFR image for the AC tumor is displayed in inset to show the low intensity of specific EGFR staining. Magnification ×20 Top row: AC patient (testing cohort with a score of 0.972 Bottom row: SCC patient (testing cohort) with score of 0.00005

FIG. 8. Three dimensional plot of principal component analysis. Principal component analysis of the original 24-dimensional data revealed that AS tumors (green squares) did not form a distinct group but were equally mixed in the AC (red squares) or SCC (blue squares) groups.

FIG. 9. Three dimensional plot of rotated components (rotation method: Varimax) Rotations were used to better align the directions of the factors with the original variables so that the factors may be more interpretable. The Varimax method tries to make elements of this matrix go toward 1 or 0 to show the clustering of the variables.

FIG. 10. Parametric correlations among the 24 proteins for adenocarcinoma and squamous cell carcinoma patients.

FIG. 11. Antibody Validation and Assay Reproducibility.

### DETAILED DESCRIPTION OF THE INVENTION

The invention provides a method for determining the histotype of a non-small cell lung cancer tumor which comprises: (a) determining the levels of expression of each of thyroid transcription factor-1, cytokeratin-5, cytokeratin-13 and epidermal growth factor receptor in a sample from the non-small cell lung cancer tumor; (b) calculating a score based on the levels of expression determined in step (a); and (c) comparing the score obtained in step (b) with a predetermined reference score associated with histotypes of non-small cell lung cancer; wherein the tumor is determined to be an adenocarcinoma if the score obtained in (b) is greater than the predetermined reference score and wherein the tumor is determined to be a squamous cell carcinoma if the score obtained in (b) is less than the predetermined reference score.

The levels of expression of thyroid transcription factor-1, cytokeratin-5, cytokeratin-13 and epidermal growth factor receptor may be determined using an automated pathology system.

The levels of expression of thyroid transcription factor-1, cytokeratin-5, cytokeratin-13 and epidermal growth factor receptor may be determined by a quantitative image analysis procedure.

Numerous quantitative image analysis procedures are known in the art. An example of a quantitative image analysis procedures that may be used to determine the level of expression include AQUA® procedures, as described in issued U.S. Pat. No. 7,219,016, and in U.S Patent Application Publication No. 2009/0034923, which are incorporated by reference into this application in its entirety.

As used in this application, the term sample can refer to various kinds of samples, for example, a tissue sample and a cytology specimen.

The tissue sample may be a fixed tissue section.

The method of this invention may be used in formalin-fixed paraffin-embedded FFPE sections, fine-needle aspirates and other histological sample types.

The method of this invention may be applied to tumor material obtained by surgical biopsy, bronchoscopic biopsies and fine-needle aspirations.

The invention provides a method for determining whether a non-small cell lung cancer tumor is an adenocarcinoma which comprises (a) determining the levels of expression of each of thyroid transcription factor-1, cytokeratin-S, cytokeratin-13 and epidermal growth factor receptor in a sample from the non-small cell lung cancer tumor; (b) calculating a score based on the levels of expression determined in step (a); and (c) comparing the score obtained in step (b) with a predetermined reference score associated with histotypes of non-small cell lung cancer; wherein the tumor is determined to be an adenocarcinoma if the score obtained in (b) is greater than the predetermined reference score.

The levels of expression of thyroid transcription factor-1, cytokeratin-5, cytokeratin-13 and epidermal growth factor receptor may be determined using an automated pathology system.

The levels of expression of thyroid transcription factor-1, cytokeratin-5, cytokeratin-13 and epidermal growth factor receptor may be determined by a quantitative image analysis procedure.

Numerous quantitative image analysis procedures are known in the art. An example of a quantitative image analysis procedures that may be used to determine the level of expression include AQUA procedures, as described in issued U.S. Pat. No. 7,219,016, and in U.S Patent Application Publication No. 2009/0034823, which are incorporated by reference into this application in its entirety.

As used in this application, the term sample can refer to various kinds of samples, for example, a tissue sample and a cytology specimen.

The tissue sample may be a fixed tissue section.

The method of this invention may be used in formalin-fixed paraffin-embedded (FFPE) sections, fine-needle aspirates and other histological sample types.

The method of this invention may be applied to tumor material obtained by surgical biopsy, bronchoscopic biopsies and fine-needle aspirations.

The invention provides a method for determining whether a non-small cell lung cancer is a squamous cell carcinoma which comprises (a) determining the levels of expression of each of thyroid transcription factor-1, cytokeratin-5, cytokeratin-13 and epidermal growth factor receptor in a sample from the non-small cell lung cancer tumor; (b) calculating a score based on the levels of expression determined in step (a); and (c) comparing the score obtained in step (b) with a predetermined reference score associated with histotypes of non-small cell lung cancer; wherein the tumor is determined to be an squamous cell carcinoma if the score obtained in (b) is less than the predetermined reference score.

The levels of expression of thyroid transcription factor-1, cytokeratin-5, cytokeratin-13 and epidermal growth factor receptor may be determined using an automated pathology system.

The levels of expression of thyroid transcription factor-1, cytokeratin-5, cytokeratin-13 and epidermal growth factor receptor may be determined by a quantitative image analysis procedure.

Numerous quantitative image analysis procedures are known in the art. An example of a quantitative image analysis procedures that may be used to determine the level of expression include AQUA® procedures, as described in issued U.S. Pat. No. 7,219,016, and in U.S Patent Application Publication No. 2009/0034823, which are incorporated by reference into this application in its entirety.

As used in this application, the term sample can refer to various kinds of samples, for example, a tissue sample and a cytology specimen.

The tissue sample may be a fixed tissue section.

The method of this invention may be used in formalin-fixed paraffin-embedded (FFPE) sections, fine-needle aspirates and other histological sample types.

The method of this invention may be applied to tumor material obtained by surgical biopsy, bronchoscopic biopsies and fine-needle aspirations.

The invention provides a method for selecting an appropriate therapeutic treatment or combination of therapeutic treatments for a patient afflicted with non-small cell lung cancer comprising (a) determining the levels of expression of each of thyroid transcription factor-1, cytokeratin-5, cytokeratin-13 and epidermal growth factor receptor in a sample of the non-small cell lung cancer tumor from the patient; (b) calculating a score based on the levels of expression determined in step (a); and (c) comparing the score obtained in step (b) with a predetermined reference score associated with histotypes of non-small cell lung cancer; (d)assigning a histotype to the non-small cell lung cancer wherein the tumor is determined to be an adenocarcinoma if the score obtained in (b) is greater than the predetermined reference score and wherein the tumor is determined to be a squamous cell carcinoma if the score obtained in (b) is less than the predetermined reference score; and (e) selecting a therapeutic treatment based on the histotype assigned in step (d).

The levels of expression of thyroid transcription factor-1, cytokeratin-5, cytokeratin-13 and epidermal growth factor receptor may be determined using an automated pathology system.

The levels of expression of thyroid transcription factor-1, cytokeratin-5, cytokeratin-13 and epidermal growth factor receptor may be determined by a quantitative image analysis procedure.

Numerous quantitative image analysis procedures are known in the art. An example of a quantitative image analysis procedures that may be used to determine the level of expression include AQUA procedures, as described in issued U.S. Pat. No. 7,219,016, and in U.S Patent Application Publication No. 2009/0034823, which are incorporated by reference into this application in its entirety.

As used in this application, the term sample can refer to various kinds of samples, for example, a tissue sample and a cytology specimen.

The tissue sample may be a fixed tissue section.

The method of this invention may be used in formalin-fixed paraffin-embedded (FFPE) sections, fine-needle aspirates and other histological sample types.

The method of this invention may be applied to tumor material obtained by surgical biopsy, bronchoscopic biopsies and fine-needle aspirations.

The therapeutic treatments of this invention may include erlotinib, gefitinib, bevacizumab, sorafenib, docetaxel, gemcitabine, pemetrexed, and cisplatin

The method of this invention may also be used in connection with patients with adenosquamous non-small cell lung cancer.

### Experimental Details

### PART I

### Abstract

Purpose: The importance of definitive histologic subclassification has significantly increased as drug trials have shown benefit or adverse effects associated with histology in non-small cell lung cancer (NSCLC). The acuity of this problem is further exacerbated by the use of minimally invasive, biopsy and cytology samples. Here we describe the development and validation of a 4-protein classifier that differentiates primary lung adenocarcinomas (AC) from squamous cell carcinomas (SCC).

Materials and methods: Quantitative immunofluorescence (AQUA®) was employed to measure and identify proteins differentially expressed between AC and SCC; 8 out of the 24 proteins studied were further analyzed in a logistic regression model following stepwise selection. An objective 4-protein classifier was generated to define likelihood of AC in a training set of 343 NSCLC patients followed by validation in 2 independent cohorts (n=197 and n=235 respectively).

Results: Statistical modeling selected TTF1, CK5, CK13 and EGFR to generate a weighted classifier in the training set and to identify the optimal cutpoint for differentiating AC from SCC tumors. Using the pathologist's final diagnosis as ground truth, the molecular test showed a sensitivity of 96% and specificity of 93%. The classifier was then validated in a blinded analysis in 2 independent cohorts; yielding a best area under the receiver operator characteristic curve of 0.989 with sensitivity and specificity of 96% and 97% respectively.

Conclusion Molecular classification of NSCLC using an objective quantitative test can be highly accurate and could be translated into a robust diagnostic platform for broad clinical application.

### Introduction

Lung cancer, predominantly non-small cell lung cancer (NSCLC), is the most common cause of death from cancer worldwide with 70-80% of the cases presenting with locally advanced or metastatic disease¹. NSCLC diagnosis and histologic subclassification is mainly performed by morphological assessment, mucin stains and immunohistochemical detection of thyroid transcription factor 1 (TTF1) and TP63². However the increased diagnostic use of cytology and very small biopsy specimens has emerged as a limiting factor for the traditional morphology-based histologic characterization of NSCLC.

Advances in targeted therapies for NSCLC revealed that histology is predictive of clinical efficacy outcomes; the most robust evidence coming from increased response rates to the EGFR tyrosine kinase inhibitors erlotinib and gefitinib³⁻⁷ and absence of hemorrhagic complications with vascular endothelial growth factor inhibitors^{8,9} and the molecule tyrosine kinase inhibitors sorafenib and sunitinib¹⁰ for tumors with adenocarcinoma (AC) histology. The association of histology with outcome of conventional chemotherapy remains highly controversial with some studies demonstrating a favorable outcome for SCC patients¹¹ and others suggesting response/survival benefit for patients with AC or non-squamous tumors¹²⁻²⁴. Recently higher response rates have been reported for AC patients treated with the pemetrexed/cisplatin doublet²⁵ highlighting the need for a standardized assay to adequately stratify patients for such treatments.

The misclassification rates for an AC diagnosis can be as high as 25% especially when comparing biopsy or cytology specimens to resections²⁶⁻³². Despite the high error rates in histologic subclassification and the unmet need for a standardized assay to adequately classify lung cancer patients by histology there is no routinely used molecular tool to distinguish between different histologic subtypes of NSCLC. Here we describe our efforts to solve this problem by developing an objective quantitative immunofluorescence test that can accurately distinguish SCC from AC histology on tissue microarrays.

One point of differentiation from other assays attempting to perform AC versus SCC differentiation is that all are technically challenging and conducted in a central laboratory. One goal is to use AQUA® analysis and its inherent standardization and reproducibility so that the assay can be provided in a decentralized fashion by multiple laboratories and each can expect to get concordant results. See, for example, the standardization described in Gustavson et al., Standardization of HER2 Immunohistochemistry in Breast Cancer by Automated Quantitative Analysis, published September 2009 in Arch. Pathol. Lab. Med., the disclosure of which is hereby incorporated by reference into this application.

### Materials and Methods

### Cohorts

Three cohorts of formalin-fixed paraffin-embedded primary NSCLC tumors were used for this study. The training set was a retrospectively collected cohort of 343 NSCLC patients diagnosed between 1991 and 2001, obtained from the Pathology Departments of Sotiria General Hospital (Athens, Greece) and Patras University General Hospital (Rion, Greece). The second cohort was retrospectively collected from 197 surgical patients from Yale-New Haven Hospital (New Haven, Conn.) between January 1995 and May 2003. The third cohort was a prospectively collected cohort of 235 NSCLC patients diagnosed between 1991 and 2001, obtained from the H. Lee Moffitt Cancer Center (Tampa, Fla.). Demographics and details on histologic subclassification and stage are shown in Supplementary Table 1. Histologic final diagnosis was based on the pathology records for all samples. The study was approved by the institutional review boards of all centers; written informed consent was obtained for each case prior to inclusion in the study.

SUPPLEMENTARY TABLE 1
Tumor and clinical characteristics of Greek (training set), Yale University (testing set).
Yale University (testing set) and Moffitt Cancer Center (validation set) Lung Cancer Cohorts

| | | | | | | Yale University | | | | Moffitt Cancer Cente | | | |
|---|---|---|---|---|---|---|---|---|---|---|---|---|---|
| | | Greek Lung Cancer Cohort | | | | Lung Cancer Cohort | | | | r Lung Cancer Cohort | | | |
| | | All patients (n = 343) | | | | All patients (n = 197) | | | | All patients (n = 235) | | | |
| Characteristic | | No. | | (%) | | No. | | (%) | | No. | | (%) | |
| Age | | | | | | | | | | | | | |
| Median (range) | | 64 (34-84) | | | | 65 (32-90) | | | | 70.5 (40-84) | | | |
| Meant ± SE | | 62.24 ± 0.49 | | | | 64.42 ± 0.75 | | | | 69.19 ± 0.55 | | | |
| Gender | | | | | | | | | | | | | |
| Male | | 302 | | 88 | | 96 | | 48.7 | | 128 | | 54.5 | |
| Female | | 41 | | 12 | | 99 | | 50.3 | | 107 | | 45.5 | |
| Missing | | | | | | 2 | | 1 | | | | | |
| Stage | | | | | | | | | | | | | |
| I | IA | 109 | 28 | 31.8 | 8.2 | 102 | 73 | 51.8 | 37.1 | 217 | 98 | 92.3 | 41.7 |
| | IB | | 81 | | 23.6 | | 29 | | 14.7 | | 119 | | 50.6 |
| II | IIA | 93 | 15 | 27.1 | 4.4 | 28 | 13 | 14.2 | 6.6 | 1 | 1 | 0.4 | 0.4 |
| | IIB | | 78 | | 22.7 | | 15 | | 7.6 | | 3 | | 1.3 |
| III | IIIA | 101 | 55 | 29.4 | 16 | 39 | 27 | 19.8 | 13.7 | 5 | 2 | 2.1 | 0.9 |
| | IIIB | | 46 | | 13.4 | | 12 | | 6.1 | | 12 | | |
| IV | | 40 | | 11.7 | | 20 | | 10.1 | | 12 | | 5.1 | |
| Missing | | | | | | 8 | | 4.1 | | | | | |

| | | | | | | |
|---|---|---|---|---|---|---|
| Histological Type | | | | | | |
| Adenocarcinoma | 138 | 40.2 | 116 | 58.9 | 121 | 51.5 |
| Squamous cell carcinoma | 167 | 18.7 | 37 | 18.8 | 86 | 36.6 |
| Large cell carcinoma | 5 | 1.5 | 18 | 9.1 | 24 | 10.2 |
| Adenosquamous | 4 | 1.2 | 14 | 7.1 | | |
| Giant cell carcinoma | 20 | 5.8 | | | | |
| Various | 10 | 2.9 | 12 | 6.1 | 4 | 1.7 |

Abbreviations:
No; number

### Tissue Microarrays

Tissue specimens were prepared in a tissue microarray (TMA) format: representative tumor areas were obtained from formalin fixed paraffin embedded (FFPE) specimens of the primary tumor and two 0.6 mm cores from each tumor block were arrayed in a recipient block. Formalin fixed paraffin embedded cell line pellets were used as controls: HT29, Calu-1, H1299, A549, SW-480, H1666, H1355, A431, HCC2279, H1819, HCC193, HC15, MCF7, H2882 and H2126 were purchased from the American Type Culture Collection (Manassas, Va.) or donated by other labs.

### Western Blotting

Equivalent amounts of protein (15 μg) were resolved by SDS-PAGE in 4-12% bis-tris gels (150V for 1 hr) and transferred at 45V for 2 hrs to a nitrocellulose membrane. Immunoblots were probed with primary antibodies (Supplementary Table 2) diluted 1/1000, followed by anti-rabbit or anti-mouse HRP conjugated secondary antibodies (Santa Cruz Biotechnology, Santa Cruz, Calif.) diluted 1/4000 and detected using enhanced chemiluminescence (GH Healthcare). β-tubulin (rabbit polyclonal, Cell Signaling Technology, Danvers, Mass.) immunoblotting was used to visualize the total protein loading.

SUPPLEMENTARY TABLE 2
Primary antibody characteristics

| | | Antigen | | | | | |
|---|---|---|---|---|---|---|---|
| Biomarker | Clone/Isotype | Retrieval | Concentration | | Incubation | Positive Control | Source |
| Cytokeratin 5 | NCL-L-CK5/m | HIAR-pH = 6 15 min | 5.2 | μg/ml | 1 hr RT | A431 cells | Novocastra, Newcastle, UK |
| Cytokeratin | DE-K13/m IgG₂ₐ, | HIAR-pH = 9 15 min | 57 | μg/ml | ON 4° C. | A431 cells | Dako, Carpinteria, CA |
| 13 | kappa | | | | | | |
| Cytokeratin | LL002/m IgG₃ | HIAR-pH = 6 15 min | 0.2 | μg/ml | ON 4° C. | A431 cells | Thermo Fisher, Fremont, CA |
| 14 | | | | | | | |
| Cytokeratin | 2D10/m IgG₁ | HIAR-pH = 6 15 min | 0.1 | μg/ml | ON 4° C. | A431 cells | Abnova, Walnut, CA |
| 17 | | | | | | | |
| EGFR | 31G7/m IgG₁ | Proteinase K | 3 | μg/ml | ON 4° C. | EGFR transfected CHO | Zymed/Invitrogen, |
| | | 5 min RT | | | | and A431 cells | Carlsbad, CA |
| HER2 | r polyclonal | HIAR-pH = 6 15 min | 25 | μg/ml | ON 4° C. | HER2 transfected CHO | Dako, Carpinteria, CA |
| | | | | | | cells | |
| HER3 | r polyclonal | HIAR-pH = 9 15 min | 0.2 | μg/ml | ON 4° C. | HER3 transfected BaF3 | Santa Cruz, Santa Cruz, |
| | | | | | | cells | CA |
| HER4 | SPM338/m IgG_{2b} | HIAR-pH = 6 15 min | 0.4 | μg/ml | ON 4° C. | HER4 transfected CHO | Santa Cruz, Santa Cruz, |
| | | | | | | cells | CA |
| AKT1 | 2H10/m | HIAR-pH = 6 15 min | 1/200* | | ON 4° C. | A431 cells | Cell Signaling, Danvers, |
| | | | | | | | MA |
| ERK | m poly | HIAR-pH = 6 15 min | 1/100* | | ON 4° C. | A431 cells | Cell Signaling, Danvers, |
| | | | | | | | MA |
| DUSP6 | 3G2/m IgG1, kappa | HIAR-pH = 6 15 min | 0.3 | μg/ml | ON 4° C. | Pancreatic carcinoma | Novus Biologicals, |
| | | | | | | | Littleton, CO |
| STAT1 | 42H3/r IgG | HIAR-pH = 6 15 min | 1/750* | | ON 4° C. | Colon carcinoma | Cell Signaling, Danvers, |
| | | | | | | | MA |
| STAT2 | Y141/r IgG | HIAR-pH = 6 15 min | 1/200* | | ON 4° C. | Breast carcinoma | Novus Biologicals, |
| | | | | | | | Littleton, CO |
| STAT3 | 124H6/m | HIAR-pH = 6 15 min | 1/500* | | ON 4° C. | H1650, HCC2279 cells | Cell Signaling, Danvers, |
| | | | | | | | MA |
| mTOR | 7C10/r | HIAR-pH = 6 15 min | 1/1000* | | ON 4° C. | Breast carcinoma, A431 | Cell Signaling, Danvers, |
| | | | | | | and H1299 cells | MA |
| pS6K | 1A5/m | HIAR-pH = 6 15 min | 1/200* | | ON 4° C. | H1299 cells | Cell Signaling, Danvers, |
| | | | | | | | MA |
| pS6 | 91B2/r IgG | HIAR-pH = 6 15 min | 1/400* | | ON 4° C. | Colon carcinoma, HCC193 | Cell Signaling, Danvers, |
| | | | | | | cells | MA |
| TTF1 | 8G7G3/m IgG1, | HIAR-pH = 6 40 min | 20 | μg/ml | ON 4° C. | H2126 cells | Dako, Carpinteria, CA |
| | kappa | | | | | | |
| E2F4 | SPM179/m IgG1, | HIAR-pH = 6 15 min | 2 | μg/ml | ON 4° C. | Tonsil | Novus Biologicals, |
| | kappa | | | | | | Littleton, CO |
| BCL2 | 124/m IgG1, kappa | HIAR-pH = 9 15 min | 2.6 | μg/ml | ON 4° C. | Lymphocytes | Dako, Carpinteria, CA |
| CC3 | 5A1/r | HIAR-pH = 6 15 min | 1/500* | | ON 4° C. | Pancreatic carcinoma | Cell Signaling, Danvers, |
| | | | | | | | MA |
| MENA | 21/m IgA | HIAR-pH = 6 15 min | 1 | μg/ml | ON 4° C. | A431 cells | BD Biosciences |
| RRM2 | 1E1/m IgG1, kappa | HIAR-pH = 6 15 min | 0.05 | μg/ml | ON 4° C. | Pancreatic carcinoma | Novus Biologicals, |
| | | | | | | | Littleton, CO |
| PTEN | 6H2.1/m IgG_{2b}, | HIAR-pH = 9 15 min | 0.96 | μg/ml | ON 4° C. | H1666 cells | Dako, Carpinteria, CA |
| | kappa | | | | | | |

Abbreviations:
m; mouse,
r; rabbit,
poly; polyclonal,
HIAR; heat induced antigen retrieval,
RT; room temperature,
ON; overnight
*antibody concentration was not provided by the manufacturer
Proteins that significantly differ in expression between adenocarcinomas and squamous cell carcinomas in italics, proteins of the 4-protein assay in bold

### Quantitative Immunofluorescence

The arrays were deparaffinized with xylene, rehydrated and antigen-retrieved by pressure cooking for 15 minutes in 10 mM citrate (pH=6) or 10 mM Tris/1 mM EDTA buffer (pH=9) for all primary antibodies but epidermal growth factor receptor (EGFR); proteinase K (Dako, Carpinteria, Calif.) enzymatic digestion was performed instead (Supplementary Table 2). Slides were pre-incubated with 0.3% bovine serum albumin (BSA) in 0.1M tris-buffered saline (TBS, pH=8) for 30 minutes at room temperature. Slides were then incubated with a cocktail of the primary antibody (Supplementary Table 2) and a mouse monoclonal anti-human cytokeratin antibody (clone AE1/AE3, M3515, Dako, Carpinteria, Calif.) or a wide-spectrum rabbit anti-cow cytokeratin antibody (Z0622, Dako, Carpinteria, Calif.) diluted 1:100 in BSA/TBS overnight at 4° C. This was followed by an 1-hour incubation with Alexa 546-conjugated goat anti-mouse secondary antibody (A11003, Molecular Probes, Eugene, Oreg.) diluted 1:100 in rabbit EnVision reagent (K4003, Dako, Carpinteria, Calif.) or Alexa 546-conjugated goat anti-rabbit secondary antibody (A11010, Molecular Probes, Eugene, Oreg.) diluted 1:100 in mouse EnVision reagent (K4001, Dako, Carpinteria, Calif.). Cyanine 5 (Cy5) directly conjugated to tyramide (FP1117, Perkin-Elmer, Boston, Mass.) at a 1:50 dilution was used as the fluorescent chromagen for target detection. Prolong mounting medium (ProLong Gold, P36931, Molecular Probes, Eugene, Oreg.) containing 4′,6-Diamidino-2-phenylindole (DAPI) was used to identify tissue nuclei. Serial sections of a smaller specialized NSCLC TMA (NSCLC control array) were stained aside all cohorts to confirm assay reproducibility. Positive controls used are described in detail in Supplementary Table 2. Negative control sections, in which the primary antibody was omitted, were used for each immunostaining run.

### Image Collection and Analysis

Automated Quantitative Analysis (AQUA®) allows exact measurement of protein concentration within subcellular compartments, as described in detail elsewhere³³. In brief, a series of high resolution monochromatic images were captured by the PM-2000™ digital imaging microscopy instrument (HistoRx, New Haven, Conn.). For each histospot, in- and out-of-focus images were obtained using the signal from the DAPI, cytokeratin-Alexa 546 and target-Cy5 channel. Target proteins were measured using a channel with emission maxima above 620 nm, in order to minimize tissue autofluorescence. Tumor was distinguished from stromal and non-stromal elements by creating an epithelial tumor “mask” from the cytokeratin signal. This created a binary mask (each pixel being either “on” or “off”) on the basis of an intensity threshold set by visual inspection of histospots. AQUA score of target proteins in the tumor mask and nuclear compartment (for TTF1 and E2F4) were calculated by dividing the target compartment pixel intensities by the area of the compartment within they were measured. AQUA scores were normalized to the exposure time and bit depth at which the images were captured, allowing scores collected at different exposure times to be directly comparable. Specimens with less that 5% tumor area per spot were not included in automated quantitative analysis for not being representative of the corresponding tumor specimen.

The image collection and analysis can also be accomplished using clustering AQUA® software, as described in U.S. Patent Application Publication No. 20090034823, entitled Compartment Segregation by Pixel Characterization Using Image Data Clustering, the contents of which is hereby incorporated by reference into this application, and as described in Gustavson at al., Development of an unsupervised pixel-based clustering algorithm for compartmentalization of immunohistochemical expression using Automated Quantitative Analysis, Appl. Immunohistochemical Mol. Morphol. 2009 July; 1794):329-37, the contents of which is hereby incorporated by reference into this application. This method and system uses autoexposure and is done automatically instead of by visual inspection of histospots.

Statistical analysis

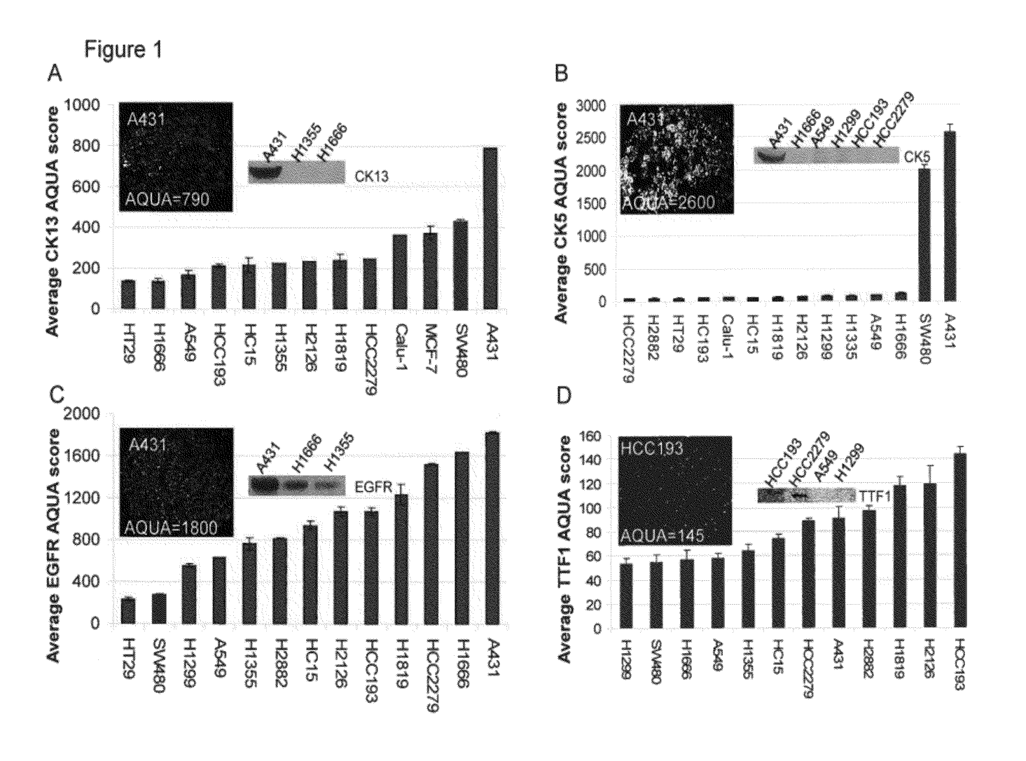

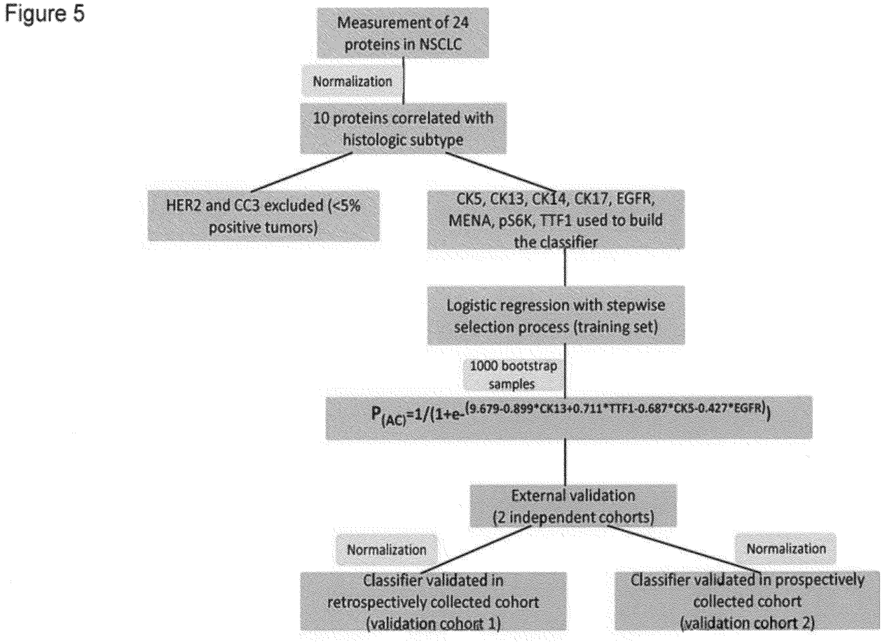

AQUA scores were Log2 normalized and scores of the validation sets were further normalized for run to run variability. Missing values were tested by Little's test for missing complete at random; cases with missing values (n=45, n=40 and n=78 for cohorts 1, 2 and 3 respectively) were excluded from analysis. Pearson's correlation coefficient (R) was used to assess the correlation between AQUA scores from redundant tumor cores as well as the same cores on serial cuts of the NSCLC control array. An R² greater than 0.4 was indicative of good inter- and intra-array reproducibility and thus the average values for all target proteins AQUA scores from duplicate samples were calculated and treated as independent continuous variables. Associations among all proteins in ACs, SCCs and adenosquamous cell carcinomas (AS) were analyzed using the Spearman rank test. Differences in biomarker expression between ACs and SCCs were assessed with the Wilcoxon sum rank test; proteins differentially expressed at a level of significance of 0.05 in more than 5% of the tumor specimens were further analyzed in a nominal logistic regression model following a backward elimination stepwise selection process³⁴ (FIG. 1). The probability of outcome of interest (prediction of AC histology) was calculated as a linear combination of the protein AQUA scores weighted by the regression estimates as follows: P_{(AC)}=1/(1+e^{-(α+β1*x1+βν*xν)}). The likelihood ratio test was used as an indicator of the model fit and the Wald statistic was used to test the significance of individual parameters. To address the issue of model over fitting 1000 bootstrap samples were generated and a backward elimination logistic regression model was developed for each bootstrap sample; the final multivariable model included those variables that were significant at the 0.05 level. The final classifier was applied to the testing and validation cohorts. The receiver operating characteristic (ROC) curve method was applied to show the sensitivity and specificity for various cut off values on scores of the training and validation sets; the Jouden index (J) was used to measure overall diagnostic effectiveness and select the cut-off score with the optimal differentiating ability between AC and SCC^{36,37}. An overview of the classifier development and validation is shown in FIG. 5. Principal component analysis (principal component 1-PC1, PC2, PC3 and PC4 set as TTF1, CK5, CK13 and EGFR respectively) was used to reduce the dimensionality and identify a small number of factors that explain most of the variance observed in the adenosquamous (AS) group. Rotations by the Varimax method were used to better align the directions of the components with the original variables and show the clustering of variables. All p values were based on two-sided testing and differences were considered significant at p<0.05. All statistical analyses were done using the SPSS software program (version 13.0 for Windows, SPSS Inc., Chicago, Ill.) and the R-statistics software (version 2.9.0).

Specimen score

Average TTF1, CK5, CK13 and EGFR scores from redundant cores were normalized for run-to-run variability, log2 transformed and a score was calculated only for tumors with all 4 measurements available using the formula score=1/(1+e−^{(9.679-0.899*CK13+0.711*TTF1-0.687*CK5-0.427*EGFR)}).

### Results

### Antibody Validation and Assay Reproducibility

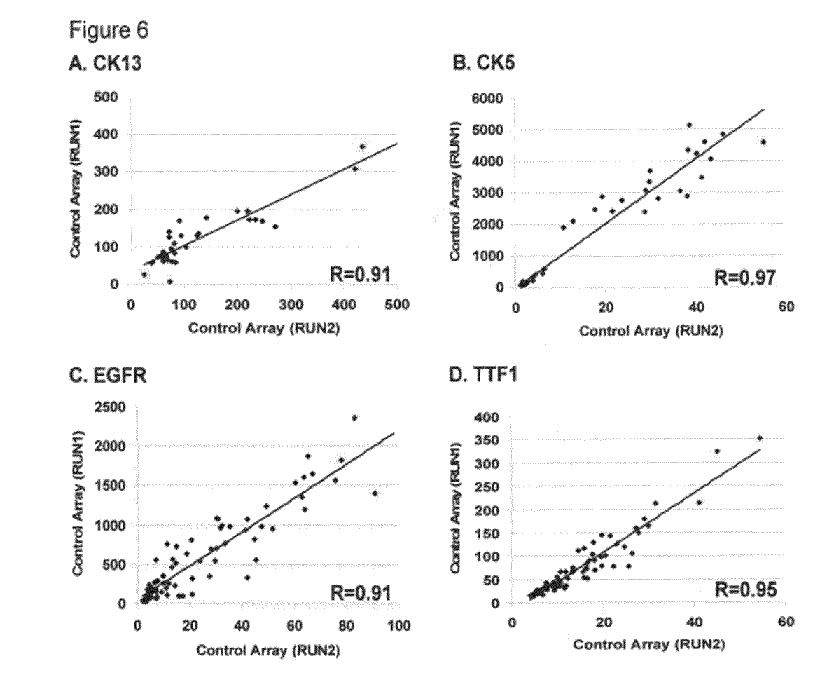

Total protein levels were independently detected by AQUA analysis and WB in cell line controls and A431, H1299, HT29, H1666, H1355, A549, HC15, H2126, H1819, HCC2279, Calu-1, MCF7, SW-480, H2882 and HCC193 cell lines showed the same range of protein expression on both AQUA and WB analysis (FIG. 1). AQUA scores were normalized for run to run variability based on the control TMA standard curve. Evaluation of the assay reproducibility did not reveal significant differences between serial sections of the NSCLC control array stained in each IHC run (Pearson's R=0.91-0.97 for all runs, p<0.0001; FIG. 6).

### Identification of Predictors of Histology

We used automated quantitative analysis to identify biomarkers that differentiate AC from SCC. Among 24 proteins assessed (Supplementary Table 2) CK5, CK13, EGFR, MENA, CK14, Cleaved Caspase 3 and CK17 were expressed at significantly higher levels in SCC and TTF1, HER2 and phospho p70 S6K were significantly higher in ACs (Table 1). These proteins were tested for intra-tumor heterogeneity by comparing AQUA scores of redundant cores. We found a highly significant correlation for all proteins analyzed (Pearson's R=0.91, p<0.0001, R=0.92, p<0.0001, R=0.86, p<0.0001, R=0.86, p<0.0001, R=0.88, p<0.0001, R=0.85, p<0.0001, R=0.63, p=0.001, R=0.81, p<0.0001, for EGFR, CK5, CK13, CK14, CK17, TTF1, MENA and phospho p70 S6K respectively). HER2 and cleaved caspase 3 were excluded from subsequent analysis because of less than 5% positive specimens; cases with values missing complete at random (Little's test p=0.6) were also excluded from the analysis.

TABLE 1
Differential expression of the 8 selected proteins in adenocarcinomas
versus squamous cell carcinomas in the training cohort

| | | | Squamous cell | | |
|---|---|---|---|---|---|
| | Adenocarcinomas | | carcinomas | | |
| Protein | No | Mean rank | No | Mean rank | p value* |
| Cytokeratin 5 | 134 | 92.32 | 163 | 195.60 | <0.0001 |
| Cytokeratin 13 | 137 | 120.66 | 162 | 174.81 | <0.0001 |
| Cytokeratin 14 | 137 | 116.58 | 166 | 181.23 | <0.0001 |
| Cytokeratin 17 | 137 | 111.03 | 164 | 184.39 | <0.0001 |
| TTF1 | 131 | 175.69 | 157 | 118.48 | <0.0001 |
| EGFR | 134 | 135.75 | 162 | 159.05 | 0.02 |
| MENA | 132 | 136.65 | 160 | 154.63 | 0.05 |
| Phospho p70 S6K | 137 | 163.92 | 159 | 135.21 | 0.004 |

Abbreviations:
No; number
*Differences in AQUA scores between adenocarcinomas and squamous cell carcinomas were evaluated by the Wilcoxon rank sum test

### Development of the Molecular Classifier (Training Cohort)

CK5, CK13, CK14, CK17, EGFR, MENA, phospho p70 S6K and TTF1 continuous AQUA scores were then incorporated in a multivariate nominal logistic regression model following a backward elimination stepwise selection process for each of the 1000 bootstrap samples; the probability of prediction of AC histology was calculated as linear combination of TTF1, CK5, CK13 and EGFR log2 normalized AQUA scores weighted as follows: P_{(AC)}=1/(1+e−^{(9.679-0.899*CK13+0.711*TTF1-0.687*CK5-0.427*EGFR)})

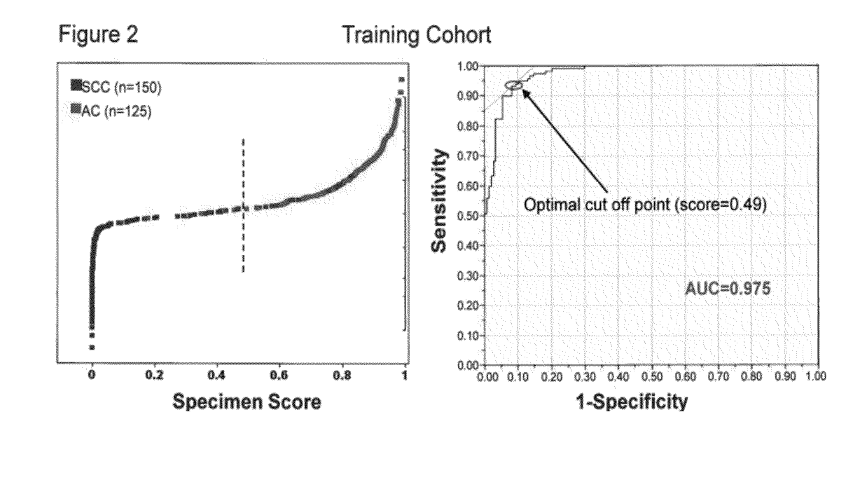

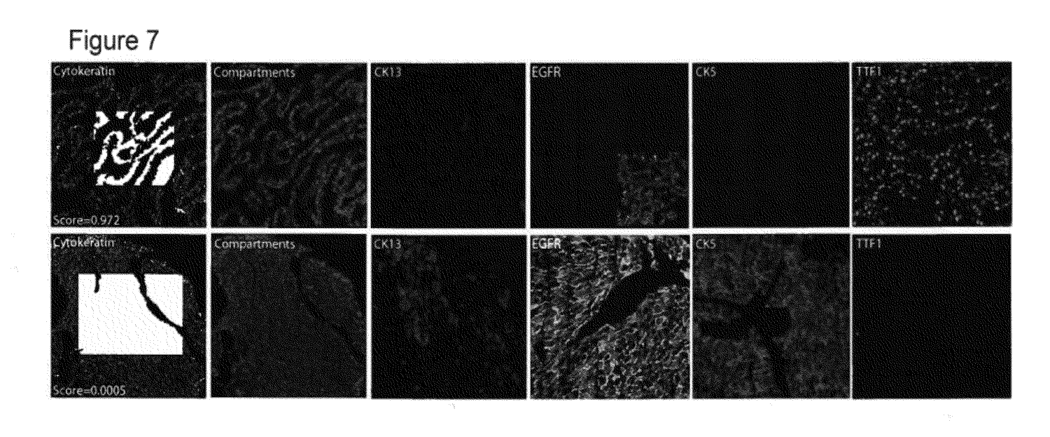

Each case of the training cohort (Greek Lung Cancer Cohort) received a score ranging from 0.00004 to 0.987 (FIG. 2); representative staining patterns for AC and SCC tumors of the training cohort are shown in FIG. 7. The Youden index (J) calculated for each score peaked for a score value of 0.49 (J=0.85) indicating a large diagnostic effectiveness; this was further used as the optimal cut off point for differentiating AC from SCC tumors. Sensitivity and specificity were 96% and 93.3% respectively with an area under the curve of 0.975 (Table 2). Based on these findings and after performing internal validation we established our diagnostic platform and used the score of 0.49 as a cut off point for the optimal AC vs. SCC classification.

TABLE 2
Classification of NSCLC specimens by the 4-protein test
Assay Classification

| Histologic Diagnosis | AC (score≧0.49) | SCC (score<0.49) |
|---|---|---|
| Training Cohort | | |
| AC | 120 | 5 |
| SCC | 10 | 140 |
| Testing Cohort | | |
| AC | 86 | 7 |
| SCC | 1 | 29 |
| Validation Cohort | | |
| AC | 72 | 3 |
| SCC | 2 | 59 |

Abbreviations:
AC; adenocarcinoma,
SCC; squamous cell carcinoma

### Classifier Validation (Validation Cohorts)

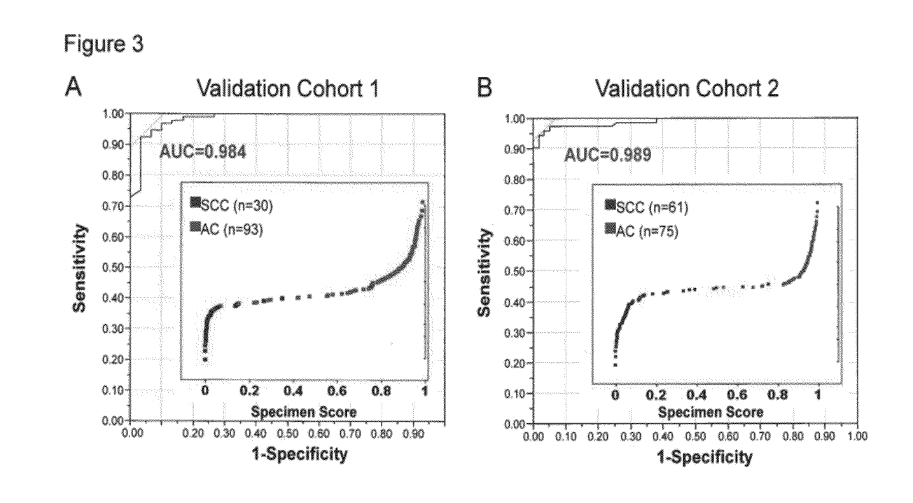

The power of the molecular classifier was tested in a retrospectively collected cohort of 197 NSCLC patients (Yale University Lung Cancer Cohort); specimens scores were measured in a blinded analysis as specified above (FIG. 3A). Eighty-six AC samples had a score of 0.49-0.99 whereas 29 SCC samples had a score of 0-0.489 without any overlap; seven AC (7.5%) and one SCC (3%) specimens were misclassified respectively (Table2). With the cut off point for classification between AC and SCC set at 0.49 our test had a sensitivity of 92%, specificity of 97% and area under the curve (AUC) equal to 0.984 (FIG. 3A). In order to test the robustness and reproducibility of our diagnostic platform we further validated the assay in a prospectively collected cohort of 235 NSCLC patients (Moffitt Cancer Center Lung Cancer Cohort). AC scores ranged from 0.49 to 0.99 and SCC scores from 0.0003 to 0.489 with the exception of 3 (4%) AC cases and 2 (3.2%) SCC misclassified specimens (Table 2). The sensitivity and specificity of identifying an AC were 96% and 97% respectively; the area under the ROC curve was 0.989 (FIG. 3B).

### Classification of Other Histologic Subtypes

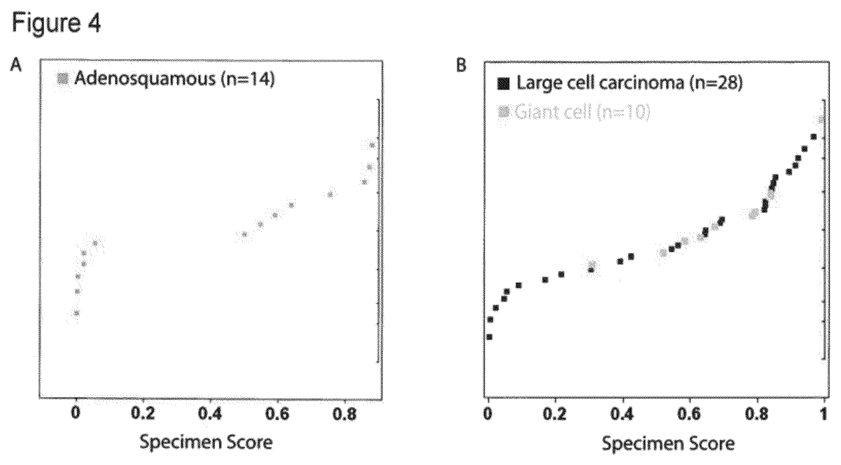

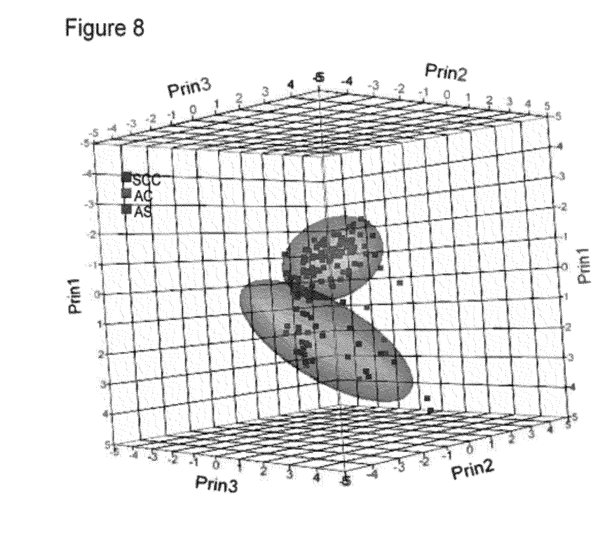

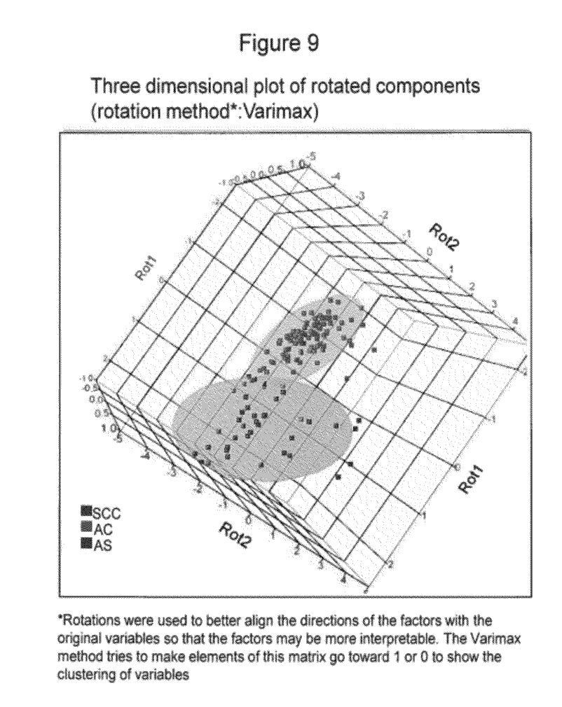

Given the exceptionally high histology predictive value of our assay we explored the use of our classifier in other histologic subtypes. We hypothesized that AS tumors were more likely to be AC-like or SCC-like rather than a distinct entity. Eight (57%) and 6 (43%) of the tumors with a morphological diagnosis of AS carcinoma showed an expression profile similar to AC and SCC respectively when the 4-protein classifier was applied (FIG. 4A). In parallel we performed principal components analysis to identify the most prominent directions of the original 24-dimensional data; interestingly enough AS did not form a distinct group but were equally mixed within the well defined AC and SCC groups for both un-rotated and rotated solutions (FIG. 8). We further applied our classifier to LCC (FIG. 4B) and giant cell carcinomas; one LCC specimen described as LCC-focally SCC had a score of 0.15 and was thus classified as a SCC however scores spanned the full range of 0-1 without preferentially clustering close to any end of the axis.

### Discussion

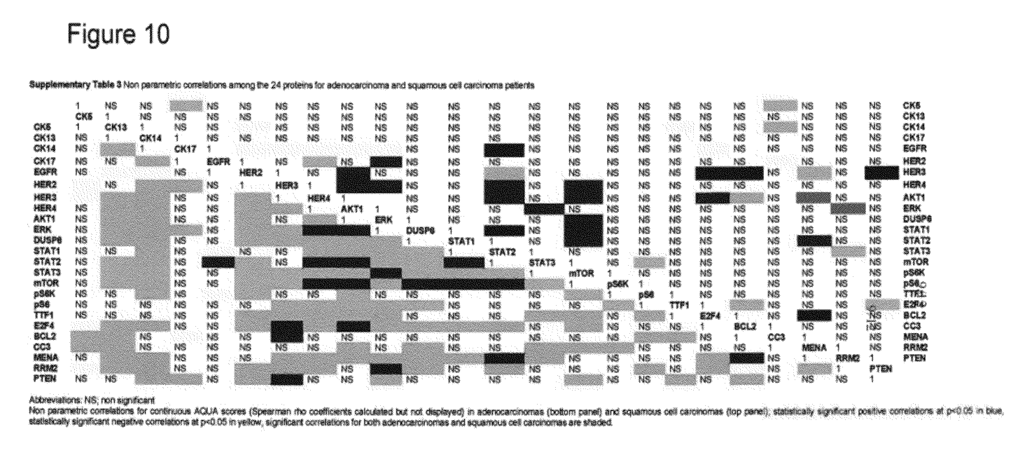

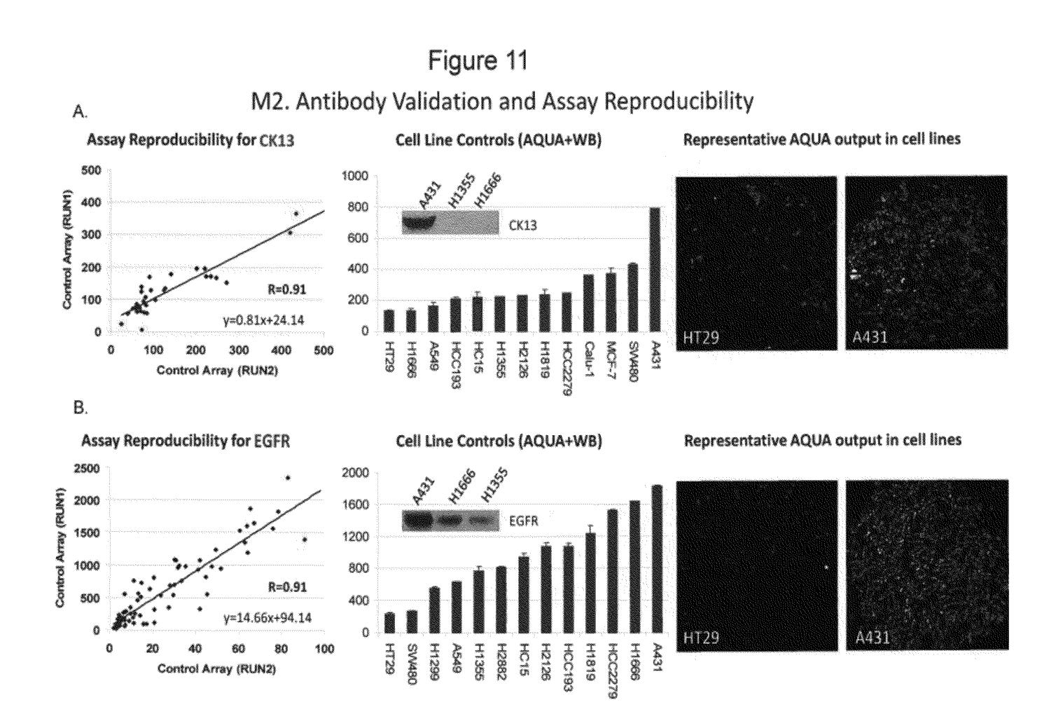

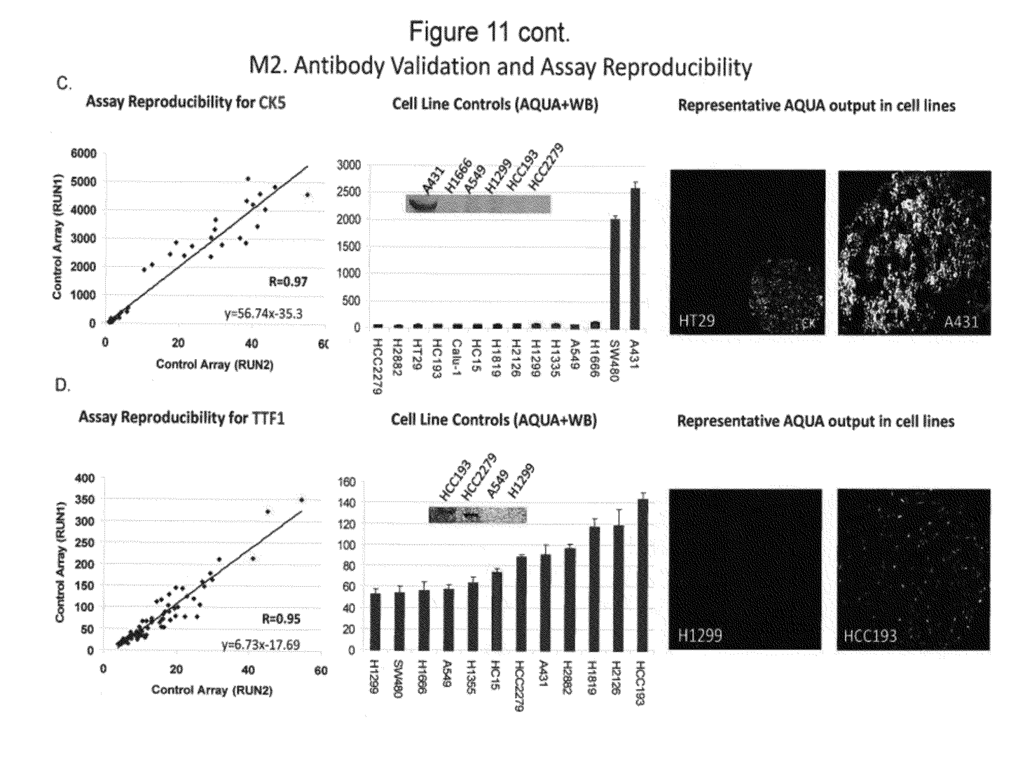

Gene expression profiling³⁸, proteomic^{39,40}, and IHC⁴¹⁻⁴⁷ studies reveal systematic differences between AC and SCC; rendering the NSCLC subclassification a matter of distinct biomarker profiles rather than a simplistic dichotomization between AC and SCC based on morphological assessment. Here we investigated the differential expression of 24 biomarkers and found proteins highly associated with NSCLC histological subtypes; interestingly correlations among proteins studied differed significantly between ACs and SCCs (FIG. 10). We found a high correlation of EGFR, CK5 and CK13 with SCC and TTF1 with AC histology; this is consistent with previous findings^{41,42,44,46,48,49}. We believe that rather than stratifying based strictly on morphological features to AC or SCC, NSCLC tumors, especially morphologically challenging tumors, can be classified on the basis of their biologic properties. However, this concept remains to be tested in prospective, randomized, controlled clinical trials. Our preliminary analysis on AS specimens revealed that these tumors did not form a distinct group but could be classified either as AC or SCC. Our test was unable to accurately classify LCC or giant cell tumors, suggesting a biologically unrelated disease matching the distinct morphology of these less common tumors.

NSCLC is routinely classified based on morphological features. The LCC diagnosis is often a diagnosis of exclusion and the criteria to make a histologic diagnosis of LCC are somewhat non-specific. One study showed that modified diagnostic criteria indicate that some tumors previously classified as large cell carcinomas could fell in the category of undifferentiated AC or SCC⁵¹. In contrast, the morphologic criteria for ACs, including glandular structures with distinct acinar, papillary, solid, bronchioloalveolar or mixed histological patterns⁵⁰ are more well defined. Similarly, SCCs are morphologically well defined including variably keratinizing tumors with a necrotic or inflammatory component⁵⁰. The sensitivity and specificity for an AC diagnosis has been studied by investigating the agreement between original and review diagnosis and has been found to be as low as 80.8% and 84.4% respectively^{26-29,52}. A case-control study assessing the agreement between regional and central pathologists demonstrated an 81% observed agreement for AC diagnosis³¹. Comparisons between pre-operative bronchial biopsies and matched resections revealed an overall concordance of 77% for AC diagnosis³¹. However the rate for correct pre-operative histologic subclassification was only 35% when bronchial biopsies and cytology specimens were used³².

To adjudicate difficult cases, the pathologist often uses immunohistochemistry. The panel routinely used to aid morphological diagnosis and differentiate between AC and SCC consists of TTF1 and p63; the former identifying ACs and the later SCCs. However the combination of those markers has a relatively low sensitivity since TTF1 is expressed in about 85% of primary lung ACs^{2,44} and 32% of SCCs⁴⁴ whereas p63 expression has been reported for 30% of ACs⁵³. This system of classification using TTF1/p63 has been shown to have a sensitivity of 74.6% leaving 14-37% of NSCLC tumors unclassified⁵⁴. Ring et al developed a more sophisticated approach using a 5 protein (TRIM29, CEACAM5, SLC7A5, MUC1 and CK5/6) test combined with a semi-qualitative scoring system and arbitrary cutpoints for biomarker positivity. This assay had an 88.6% sensitivity and an 85.9% specificity with 12% of cases remaining unclassified⁵⁴. Although our assay is similar since it a based on a series of immunoassays, we believe we achieve greater accuracy since our quantitative test allows more precise measurement of expression and more selective cut points with more histotype specific proteins.

Micro-RNA assays represents another approach to NSCLC classification. The microRNA miR-205 has been shown to be an accurate marker for SCC histology as a single predictor⁵⁵ as well as one of a set of 6 microRNAs⁵⁶. The sensitivity, specificity and area under the curve for the miR-205 assay are 96%, 90% and 0.96 respectively when using cutoffs for high confidence classification. While this test represents an alternative molecular mechanism of classification, and confirms the biological differentiation we observed, it is a destructive test. That is, the material is ground up and extracted adding the question of tissue sampled into the mix; analysis of specimens that contain very little tumor and a lot of inflammatory cells or other non-tumor tissue would result in inaccurate readings.

However, this work is also subject to a series of limitations. Specifically, in all cases we used only fields collected from tissue microarray spots. While this provides a compelling result, TMAs are never used in routine diagnostics, and furthermore, the number of fields of view examined by TMAs are likely substantially fewer than is done in a routine pathology setting. However, TMA field-of-view numbers are not dissimilar from cytology specimens where classification is particularly challenging. We are currently testing the assay on cytology specimens and small fragment tissue biopsy specimens. Another limitation is that our cases were retrospectively collected from a handful of institutions. In the future, we hope a cooperative group might undertake a multi-site prospective clinical trial to validate this assay. Finally, our assay is not specifically linked to any therapeutic. While this is not a true limitation of the test, trials linking this assay to a specific drug may improve the connection between histotype and response and/or occurrence of adverse effects.

Recent research has shown that this work can be applied in cytology specimens. The most difficult application is when there is only a very small amount and one of the potential uses that distinguishes this invention is that it can be applied in cytology specimens instead of chunks of tumor.

In conclusion, we have developed a highly reproducible, objective, easily applicable quantitative immuno-assay based test for subclassification of lung cancer. Once it is validated in a prospective manner in biopsies and cytology specimens, this test could easily be translated into a robust diagnostic platform for broad clinical application. Moreover, we believe that identifying the histologic and molecular properties of tumors more likely to respond to specific treatments will result in the fine tuning of personalized therapy towards the goal of maximizing survival outcomes for lung cancer patients.

### SYNOPSIS

Background: Non-small cell lung cancer (NSCLC) diagnosis is mainly performed by morphological assessment, with TTF1, CK5/6 and TP63 being two conventionally used markers for NSCLC sub classification.

However, there has been increased use of fine needle aspiration (FNA) material for NSCLC diagnosis. In addition, the present methods for NSCLC classification are not reproducible. The Iowa Cancer Registry (Field et al; 2004, J. Nat'l Cancer Institute) demonstrated 80.8% sensitivity and 84.4% specificity for adenocarcinoma (AC) diagnosis. The Missouri Cancer Registry (Brownson et al; 1995, Cancer) demonstrated 65.6% agreement rate between original and review diagnosis for adenocarcinomas. The Germany Registry (Stang et al; 2005, Lung Cancer) demonstrated 81% agreement between regional and central pathologists for adenocarcinoma.

Histology can determine targeted and conventional combined modality treatment (CMT). Erlotinib and gefitinib treatments have a better response in the AC group (Lynch et al., 2004, N. Engl. J. Med.; Paex et al, 2004, Science; Shepherd et al., 2005, N. Engl. J. Med.). Squamous cell carcinomas (SCCs) are not eligible for bevacizumab (Johnson at al., 2004, J. Clin. Oncol.; Sandler et al, 2006, N. Engl. J. Med.). Sorafenib and sunitinib show an increase morbidity in the SCC group (Socinski at al., 2007; Chest, Gridelli at al.; 2007, Oncologist). Docetaxel in combination with gemcitabine have a better response rate in the AC group and pemetrexed in combination with cisplatin have a better response rate in the AC group (Scagliotti at al., 2008, J. Clin. Oncol.). There is currently no standardized assay to stratify patients for such treatments.

There are a number of current molecular tools for NSCLC classification. First, both gene (Weir et al., 2007, Nature) expression profiling and proteomic (Yanagisawa et al., 2003, The Lancet; Seike at al., 2005, Proteomics) studies reveal systematic differences between AC and SCC. Such studies are limited by lack of reproducibility and independent validation of the results as well as time consuming and expensive methodology. Another is micro-RNA based assays—Has-miR-205 has been shown to be an accurate marker for SCC as a single predictor (sensitivity 96% and specificity 90%) (Lebanony et al; 2009, J. Clin. Oncol.) as well as one of a set of six microRNAs (accuracy 81%) (Yanaihara et al; 2006, Cancer Cell). Another is Pulmotype—5 protein (TRIM29, CEACAM5, SLC7A5, MUC1 and CK5/6) chromagenic IHC based test, semi-qualitative antibody scoring, arbitrary cutpoint selection for positivity, assay reproducibility no shown, 3.5% misclassification rate, 11% of cases remain unclassified (Ring et al; 2009, Modern Path.).

### Objectives:

A. Develop a quantitative, reproducible and easily applicable protein test of NSCLC sub-classification.

B. Assess adenosquamous (AS) classification—is AS AC-like or SCC-like rather than a distinct entity.

### Materials and Methods

### M1.Cohorts

Tissue microarray (TMA) format, 2× redundancy per patient.

Training set YTMA140/Greek Lung Cancer TMA (AC=137, SCC=167).

Testing set YTMA79/Yale Lung Cancer TMA (AC=117, SCC=37, AS=13).

### M2. Antibody Validation and Assay Reproducibility

Correct for run to run variability (control array standard curve).

The assessment of protein expression in cell line controls is done by two independent methods, (1) AQUA and (2) Western Blot (WB).

### M3. Automated Quantitative Analysis in NSCLC

Intratumor heterogeneity was assessed by comparing AQUA scores of redundant cores. A high significant correlation was observed for all proteins analyzed (Pearson's R=0.91, p<0.0001, R=0.92, p<0.0001, R=0.86, p<0.0001, R=0.86, p<0.0001, R=0.88, p<0.0001, R=0.85, p<0.0001, R=0.63, p=0.001, R=0.81, p<0.0001, for EGFR, CK5, CK13, CK14, CK17, TTF1, MENA and phospho p70 S6K respectively). Specimens with less than 5% tumor area spot were not included in the automated quantitative analysis for not being representative of the corresponding tumor specimen.

### M4. Biomarker Selection

Ten proteins (among 27 tested) were selected as for being differentially expressed between AC and SCC: CK5, TTF1, CK13, EGFR, phospho p70 S6K, MENA, CK14, HER2, Cleaved Caspase 3 and CK17 (Wilcoxon test, significance at the 0.05 level). TTF1, HER2, phospho p70 S6K and CK5, CK13, CK14, CK17, EGFR, MENA, cleaved caspase 3 were over-expressed in ACs and SCCs respectively. HER2 and cleaved caspase 3 were excluded because of less than 5% positive specimens. CK5, CK13, CK14, CK17, EGFR, MENA, phospho p70 S6K and TTF1 were further used to generate a logistic regression model for histology prediction.

### M5. Statistical Analysis

Data Normalization: All AQUA scores (training and testing sets) were Log2 normalized, scores of the testing set were further normalized for run to run variability (a NSCLC control array was used to construct a standard curve for each protein).

Missing data: 2.9%, 2.6%, 5% and 1.9% cases with values missing completely at random (Little's test, p=0.6, SPSS version 13.0 for Windows) for EGFR, CK5, TTF1 and CK13 respectively.

Model construction-training set: Nominal logistic regression-backward elimination selection process (R version 2.0.0). The probability of outcome of interest (here prediction of AC histology) is a linear combination of the protein AQUA scores weighted by the regression estimates and is calculated as follows: P=1/(1+e^{-(α+β}1^{*x}1^{+β}2^{*x}2^{+ . . . +β}ν^{*x}ν⁾).

Model over fitting: 1000 bootstrap samples were generated and a backward elimination logistic regression model was developed for each bootstrap sample. The final multivariable prognostic model included those variables that were significant at the 0.05 level.

Model evaluation on testing set: Final prediction model generated above was applied to the testing cohort and each case received a score ranging from 0.0000392 to 0.9878.

Sensitivity and specificity: The receiver operating characteristic (ROC) curve method was applied to show the sensitivity and specificity for various cut off values on scores of the testing set. A cut off point of 0.4 was selected in order to maximize the specificity of the model. AC histology was considered as true positive.

Validation of predicted probability-validation set: A third array will be used to further test the model and validate the selected cut off point for AC prediction.

### Results—YTMA 140 Training Set

TABLE 1
First full model

| Model | −LogLikelihood | DF | ChiSquare | Prob > ChiSq |
|---|---|---|---|---|
| Difference | 95.71859 | 8 | 191.4372 | <.0001* |
| Full | 89.91746 | | | |
| Reduced | 185.63605 | | | |
| RSquare (U) | 0.5156 | | | |
| Observations | 269 | | | |
| (or Sum | | | | |
| Wgts) | | | | |
| Term | Estimate | Std Error | ChiSquare | Prob > ChiSq |
| Intercept | 5.88994374 | 5.2809191 | 1.24 | 0.2647 |
| egfrLog2 | −0.3563629 | 0.1454262 | 6.00 | 0.0143* |
| ck14Log2 | −0.32381 | 0.2685377 | 1.45 | 0.2279 |
| ck17log2 | 0.11159179 | 0.1662074 | 0.45 | 0.5020 |
| ck5log2 | −0.626275 | 0.1211442 | 26.73 | <.0001* |
| menalog2 | −0.1929828 | 0.224161 | 0.74 | 0.3893 |
| ttf1log2 | 0.73806564 | 0.1941067 | 14.46 | 0.0001* |
| ps6log2 | 0.5873934 | 0.6016686 | 0.95 | 0.3289 |
| ck13log2 | −0.8791329 | 0.3986095 | 4.86 | 0.0274* |

For log odds of 0/1

The likelihood ratio test was used an indicator of the model fit [null hypothesis=the reduced model fits no better than fixed response rates (full model)].

Prob>ChiSq (p value) indicates the probability of getting by chance alone a Chi-square value greater than the one computed.

Parameter estimates given by the logistic regression model are shown along with the SE, Chi-square and observed significant probability for the Chi-square test.

The Wald statistic was used to test the significance of individual parameters (null hypothesis=each of the parameters is zero).

TABLE 2
Final reduced model

| Model | −LogLikelihood | DF | ChiSquare | Prob > ChiSq |
|---|---|---|---|---|
| Difference | 93.76302 | 4 | 187.526 | <.0001* |
| Full | 96.50080 | | | |
| Reduced | 190.26382 | | | |
| RSquare (U) | 0.4928 | | | |
| Observations | 276 | | | |
| (or Sum | | | | |
| Wgts) | | | | |
| Converged | | | | |
| by Gradient | | | | |
| Term | Estimate | Std Error | ChiSquare | Prob > ChiSq |
| intercept | 9.06533379 | 2.1821359 | 17.26 | <.0001* |
| ck13log2 | −0.8146192 | 0.3377968 | 5.82 | 0.0159* |
| ttf1log2 | 0.67130893 | 0.1729829 | 15.06 | 0.0001* |
| ck5log2 | −0.6852223 | 0.0980765 | 48.81 | <.0001* |
| egfrLog2 | −0.3937343 | 0.1309927 | 9.03 | 0.0026* |

For log odds of 0/1

Model equation P_{(AC)}−1/(1+e−^{9.065-0.814*CK13+0.671*TTF1-0.685*CK5-0.393*EGFR)})

1000 bootstrap samples

Model Equation P_{(AC)}−1/(1+e−^{9.679-0.899*CK13+0.711*TTF1-0.687*CK5-0.427*EGFR)})

Principal components analysis (PCA): PCA was used to reduce the dimensionality and identify a small number of factors that explain most of the variance observed in the testing set.

Adenosquamous (AS) classification is assessed to determine if AS is AC-like or SCC-like using PCA analysis. PCA was performed to identify the most prominent directions of the 28-dimensional data. Interestingly, AC and SCC clustered together however AS did not form a significant group.

### Discussion

Morphological assessment and qualitative IHC-based sub-classification of NSCLC has reached a plateau in efficacy for predicting histology.

A biomarker based histology predictor could be used on less invasive biopsy samples (i.e., bronchoscopic biopsies, FNAs).

The results raise the question whether a molecular phenotype (protein expression pattern) rather than a histological subtype could be used to predict which patients would experience an adverse event (bleeding) and select patients for bevacizumab treatment.

Patients with AS tumors were classified into AC or SCC groups according to their protein expression pattern rather than forming a distinct group. Those patients might favor from AC/SCC specific treatment strategies.

This classifier might be of prognostic value and identify candidates for targeted therapies.

### PART II

### THERAPEUTIC TREATMENTS FOR NSCLC—AC VS. SCC Pemetrexed (Alimta, Lilly)

Mechanism of Action: Pemetrexed is chemically similar to folic acid and is in the class of chemotherapy drugs called folate antimetabolites. It works by inhibiting three enzymes used in purine and pyrimidine synthesis-thymidylate_synthase (TS), dihydrofolate reductase (DHFR), and glycinamide ribonucleotide formyltransferase^{[59][60]} (GARFT). By inhibiting the formation of precursor purine and pyrimidine nucleotides, pemetrexed prevents the formation of DNA and RNA, which are required for the growth and survival of both normal cells and cancer cells.

Adenocarcinoma has a better response than squamous (but squamous can respond).

FDA approval 2/2004 for mesothelioma; 9/2008 for NSCLC 1^{st} line with cisplatin, against of locally-advanced and metastatic NSCLC, in patients with non-squamous histology.^{[61]}

### Erlotinib (OSI-774, Tarceva, Genenetech & OSI in U.S.; Roche in ROW)

Mechanism of Action: erlotinib specifically targets the epidermal growth factor receptor (EGFR) tyrosine kinase, which is highly expressed and occasionally mutated in various forms of cancer. It binds in a reversible fashion to the adenosine triphosphate (ATP) binding site of the receptor.^{[62]} For the signal to be transmitted, two members of the EGFR family need to come together to form a homodimer. These then use the molecule of ATP to autophosphorylate each other, which causes a conformational change in their intracellular structure, exposing a further binding site for binding proteins that cause a signal cascade to the nucleus. By inhibiting the ATP, autophosphorylation is not possible and the signal is stopped.

Response: It is reported that responses among patients with lung cancer are seen most often in females who were never smokers, particularly Asian women and those with adenocarcinoma cell type.

Resistance: A key issue with EGFR-directed treatments is that after a period of 8-12 months, the cancer cells become resistant to the treatment, most commonly by recruiting a mutated IGF-1 receptor to act as one of the EGFR partners in the homodimer, so forming a heterodimer.^{[63]} This allows the signal to be transmitted even in the presence of an EGFR inhibitor. Some IGR-1R inhibitors are in various stages of development (based either around tyrphostins such as AG1024 or AG538^{[64]} or pyrrolo[2,3-d]-pyrimidine derivatives such as NVP-AEW541.^{[65]}

FDA approval 11/2004 locally advanced or metastatic NSCLC, after failure of at least one other cheoRx, in combination with gemcitabine as first line.

### Bevacuzimab (Avastin, Genentech/Roche)

Bevacizumab (trade name Avastin, Genentech/Roche) is a monoclonal antibody against vascular endothelial growth factor-A (VEGF-A)^{[66]} It is used in the treatment of cancer, where it inhibits tumor growth by blocking the formation of new blood vessels (angiogenesis). Bevacizumab was the first clinically available angiogenesis inhibitor in the United States.

In 2006, the FDA approved bevacizumab for use in lung cancer in combination with standard first-line chemotherapy. A study conducted by the Eastern Cooperative Oncology Group (ECOG) demonstrated a 2-month improvement in overall survival in patients with Stage IIIb/IV non-small cell lung cancer (NSCLC). Due to the observance of severe pulmonary hemorrhage in patients with NSCLC with squamous histology in an earlier study, patients with such histology were excluded from the pivotal ECOG trial.

### Gemcitabine

Gemcitabine is a nucleoside analog used as chemotherapy. It is marketed as Gemzar by Eli Lilly and Company. Chemically gemcitabine is a nucleoside analog in which the hydrogen atoms on the 2′ carbons of deoxycytidine are replaced by fluorine atoms.

As with fluorouracil and other analogues of pyrimidines, the drug replaces one of the building blocks of nucleic acids, in this case cytidine, during DNA replication. The process arrests tumor growth, as new nucleosides cannot be attached to the “faulty” nucleoside, resulting in apoptosis.

Adeno vs. Squamous NSCLC

Historically there has been no difference in survival. New treatments on the market include Alimta and Tarceva. Adenocarcinoma has the best response and OSI and Genentech try to target this group. It is not advisable to give bevacuzimab to squamous NSCLC because of a hemorrhage risk. Squamous tends to be located more centrally in the lung—close to target blood vessels. Adenocarcinoma is more peripheral.

### Treatment Developments for NSCLC

November 2004: Tarceva (erlotinib, EGFR inhibitor, Genentech & OSI), FDA approval is line with gemcitabine; 2^{nd} failure on chemo. NSCLC-non squamous phenotype.

Gemzar (gemcitabine, nucleoside analog, Lilly)

2006: Avastin (bevacizumab, VEFG inhibitor, Genentech) FDA approval 1^{st} line with chemo. Squamous excluded from trial.

September 2008: Alimta (pemetrexed, anti-folate, Lilly) FDA approval 1^{st} line NSCLC, non squamous.

Lilly products: Adenocarcinoma is better than Alimta Squamous has better response to Gemzar.

This has driven requests of pathologists to differentiate between adeno and squamous carcinomas.

### REFERENCES

1. Jemal A, Siegel R, Ward E, et al: Cancer statistics, 2006. CA Cancer J Clin 56:106-30, 2006

2. Brambilla E, Travis W D, Colby T V, et al: The new World Health Organization classification of lung tumours. Eur Respir J 18:1059-68, 2001

3. Lynch T J, Bell D W, Sordella R, et al: Activating mutations in the epidermal growth factor receptor underlying responsiveness of non-small-cell lung cancer to gefitinib. N Engl J Med 350:2129-39, 2004

4. Paez J G, Janne P A, Lee J C, at al: EGFR mutations in lung cancer: correlation with clinical response to gefitinib therapy. Science 304:1497-500, 2004

5. Shepherd F A, Rodrigues Pereira J, Ciuleanu T, at al: Erlotinib in previously treated non-small-cell lung cancer. N Engl J Med 353:123-32, 2005

6. Fukuoka M, Yano S, Giaccone G, at al: Multi-institutional randomized phase II trial of gefitinib for previously treated patients with advanced non-small-cell lung cancer (The IDEAL 1 Trial) [corrected]. J Clin Oncol 21:2237-46, 2003

7. Kris M G, Natale R B, Herbst R S, at al: Efficacy of gefitinib, an inhibitor of the epidermal growth factor receptor tyrosine kinase, in symptomatic patients with non-small cell lung cancer: a randomized trial. Jama 290:2149-58, 2003

8. Sandler A, Gray R, Perry M C, et al: Paclitaxel-carboplatin alone or with bevacizumab for non-small-cell lung cancer. N Engl J Med 355:2542-50, 2006

9. Johnson D H, Fehrenbacher L, Novotny W F, at al: Randomized phase II trial comparing bevacizumab plus carboplatin and paclitaxel with carboplatin and paclitaxel alone in previously untreated locally advanced or metastatic non-small-cell lung cancer. J Clin Oncol 22:2184-91, 2004

10. Gridelli C, Maione P, Del Gaizo F, et al: Sorafenib and sunitinib in the treatment of advanced non-small cell lung cancer. Oncologist 12:191-200, 2007

11. Ardizzoni A, Boni L, Tiseo M, et al: Cisplatin- versus carboplatin-based chemotherapy in first-line treatment of advanced non-small-cell lung cancer: an individual patient data meta-analysis. J Natl Cancer Inst 99:847-57, 2007

12. Martins S J, Pereira J R: Clinical factors and prognosis in non-small cell lung cancer. Am J Clin Oncol 22:453-7, 1999

13. Weiss G J, Rosell R, Fossella F, et al: The impact of induction chemotherapy on the outcome of second-line therapy with pemetrexed or docetaxel in patients with advanced non-small-cell lung cancer. Ann Oncol 18:453-60, 2007

14. Shinkai T, Eguchi K, Sasaki Y, et al: A prognostic-factor risk index in advanced non-small-cell lung cancer treated with cisplatin-containing combination chemotherapy. Cancer Chemother Pharmacol 30:1-6, 1992

15. Itaya T, Yamaoto N, Ando M, at al: Influence of histological type, smoking history and chemotherapy on survival after first-line therapy in patients with advanced non-small cell lung cancer. Cancer Sci 98:226-30, 2007

16. Saynak M, Aksu G, Fayda M, et al: The results of concomitant and sequential chemoradiotherapy with cisplatin and etoposide in patients with locally advanced non-small cell lung cancer. J Buon 10:213-8, 2005

17. Finkelstein D M, Ettinger D S, Ruckdeschel J C: Long-term survivors in metastatic non-small-cell lung cancer: an Eastern Cooperative Oncology Group Study. J Clin Oncol 4:702-9, 1986

18. Fukuoka M, Masuda N, Furuse K, et al: A randomized trial in inoperable non-small-cell lung cancer: vindesine and cisplatin versus mitomycin, vindesine, and cisplatin versus etoposide and cisplatin alternating with vindesine and mitomycin. J Clin Oncol 9:606-13, 1991

19. Rapp E, Pater J L, Willan A, et al: Chemotherapy can prolong survival in patients with advanced non-small-cell lung cancer—report of a Canadian multicenter randomized trial. J Clin Oncol 6:633-41, 1988

20. Ceresoli G L, Gregorc V, Cordio S, et al: Phase II study of weekly paclitaxel as second-line therapy in patients with advanced non-small cell lung cancer. Lung Cancer 44:231-9, 2004

21. Charloux A, Hedelin G, Dietemann A, et al: Prognostic value of histology in patients with non-small cell lung cancer. Lung Cancer 17:123-34, 1997

22. LeCaer H, Barlesi F, Robinet G, et al: An open multicenter phase II trial of weekly docetaxel for advanced-stage non-small-cell lung cancer in elderly patients with significant comorbidity and/or poor performance status: The GFPC 02-02b study. Lung Cancer 57:72-8, 2007

23. Rudd R M, Gower N H, Spiro S G, et al: Gemcitabine plus carboplatin versus mitomycin, ifosfamide, and cisplatin in patients with stage IIIB or IV non-small-cell lung cancer: a phase III randomized study of the London Lung Cancer Group. J Clin Oncol 23:142-53, 2005

24. Cellerino R, Tummarello D, Guidi F, et al: A randomized trial of alternating chemotherapy versus best supportive care in advanced non-small-cell lung cancer. J Clin Oncol 9:1453-61, 1991

25. Scagliotti G V, Parikh P, von Pawel J, et al: Phase III study comparing cisplatin plus gemcitabine with cisplatin plus pemetrexed in chemotherapy-naive patients with advanced-stage non-small-cell lung cancer. J Clin Oncol 26:3543-51, 2008

26. Brownson R C, Loy T S, Ingram E, at al: Lung cancer in nonsmoking women. Histology and survival patterns. Cancer 75:29-33, 1995

27. Field R W, Smith B J, Platz C E, at al: Lung cancer histologic type in the surveillance, epidemiology, and end results registry versus independent review. J Natl Cancer Inst 96:1105-7, 2004

28. Greenberg E R, Korson R, Baker J, et al: Incidence of lung cancer by cell type: a population-based study in New Hampshire and Vermont. J Natl Cancer Inst 72:599-603, 1984

29. Kreuzer M, Muller K M, Brachner A, et al: Histopathologic findings of lung carcinoma in German uranium miners. Cancer 89:2613-21, 2000

30. Stang A, Pohlabeln H, Muller K M, at al: Diagnostic agreement in the histopathological evaluation of lung cancer tissue in a population-based case-control study. Lung Cancer 52:29-36, 2006

31. Cataluna J J, Perpina M, Greses J V, at al: Cell type accuracy of bronchial biopsy specimens in primary lung cancer. Chest 109:1199-203, 1996

32. Edwards S L, Roberts C, McKean M E, et al: Preoperative histological classification of primary lung cancer: accuracy of diagnosis and use of the non-small cell category. J Clin Pathol 53:537-40, 2000

33. Camp R L, Chung G G, Rimm D L: Automated subcellular localization and, quantification of protein expression in tissue microarrays. Nat Med 8:1323-7, 2002

34. Simon R: Roadmap for developing and validating therapeutically relevant genomic classifiers. J Clin Oncol 23:7332-41, 2005

35. Molinaro A M, Simon R, Pfeiffer R M: Prediction error estimation: a comparison of resampling methods. Bioinformatics 21:3301-7, 2005

36. Zou K H, Hall W J, Shapiro D E: Smooth non-parametric receiver operating characteristic (ROC) curves for continuous diagnostic tests. Stat Med 16:2143-56, 1997

37. Youden W: An index for rating diangostic tests. Cancer 3:32-35, 1950

38. Weir B A, Woo M S, Getz G, et al: Characterizing the cancer genome in lung adenocarcinoma. Nature 450:893-8, 2007

39. Yanagisawa K, Shyr Y, Xu B J, at al: Proteomic patterns of tumour subsets in non-small-cell lung cancer. Lancet 362:433-9, 2003

40. Seike M, Kondo T, Fujii K, et al: Proteomic signatures for histological types of lung cancer. Proteomics 5:2939-48, 2005

41. Deeb G, Wang J, Ramnath N, et al: Altered E-cadherin and epidermal growth factor receptor expressions are associated with patient survival in lung cancer: a study utilizing high-density tissue microarray and immunohistochemistry. Mod Pathol 17:430-9, 2004

42. Jeon Y K, Sung S W, Chung J H, et al: Clinicopathologic features and prognostic implications of epidermal growth factor receptor (EGFR) gene copy number and protein expression in non-small cell lung cancer. Lung Cancer 54:387-98, 2006

43. Anagnostou V K, Bepler G, Syrigos K N, et al: High expression of mammalian target of rapamycin is associated with better outcome for patients with early stage lung adenocarcinoma. Clin Cancer Res 15:4157-64, 2009

44. Anagnostou V K, Syrigos K N, Bepler G, et al: Thyroid transcription factor 1 is an independent prognostic factor for patients with stage I lung adenocarcinoma. J Clin Oncol 27:271-8, 2009

45. Ceppi P, Volante M, Saviozzi S, et al: Squamous cell carcinoma of the lung compared with other histotypes shows higher messenger RNA and protein levels for thymidylate synthase. Cancer 107:1589-96, 2006

46. Kargi A, Gurel D, Tuna B: The diagnostic value of TTF-1, CK 5/6, and p63 immunostaining in classification of lung carcinomas. Appl Immunohistochem Mol Morphol 15:415-20, 2007

47. Cappuzzo F, Tallini G, Finocchiaro G, et al: Insulin-like growth factor receptor 1 (IGF1R) expression and survival in surgically resected non-small-cell lung cancer (NSCLC) patients. Ann Oncol, 2009

48. Suo Z, Holm R, Nesland J M: Squamous cell carcinomas. An immunohistochemical study of cytokeratins and involucrin in primary and metastatic tumours. Histopathology 23:45-54, 1993

49. Hirsch F R, Scagliotti G V, Langer C J, et al: Epidermal growth factor family of receptors in preneoplasia and lung cancer: perspectives for targeted therapies. Lung Cancer 41 Suppl 1:S29-42, 2003

50. Travis W D B E, Muller-Hermelink H K, Harris C C: World Health Organization classification of tumors. Pathology and genetics of tumors of the lung, pleura, thymus and heart. Lyon, IARC Press, 2004

51. Hirsch F R, Spreafico A, Novello S, et al: The prognostic and predictive role of histology in advanced non-small cell lung cancer: a literature review. J Thorac Oncol 3:1468-81, 2008

52. Yamamoto S, Sobue P. Yamaguchi N, et al: Reproducibility of diagnosis and its influence on the distribution of lung cancer by histologic type in Osaka, Japan. Jpn J Cancer Res 91:1-8, 2000

53. Au N H, Gown A M, Cheang M, et al: P63 expression in lung carcinoma: a tissue microarray study of 408 cases. Appl Immunohistochem Mol Morphol 12:240-7, 2004

54. Ring B Z, Seitz R S, Beck R A, et al: A novel five-antibody immunohistochemical test for subclassification of lung carcinoma. Mod Pathol 22:1032-43, 2009

55. Lebanony D, Benjamin H, Gilad S. at al: Diagnostic assay based on hsa-miR-205 expression distinguishes squamous from nonsquamous non-small-cell lung carcinoma. J Clin Oncol 27:2030-7, 2009

56. Yanaihara N, Caplen N, Bowman E, at al: Unique microRNA molecular profiles in lung cancer diagnosis and prognosis. Cancer Cell 9:189-98, 2006

57. Gustayson et al., Development of an unsupervised pixel-based clustering algorithm for compartmentalization of immunohistochemical expression using Automated Quantitative Analysis, Appl. Immunohistochemical Mol. Morphol. 2009 July; 1794):329-37.

58. Gustayson et al., Standardization of HER2 Immunohistochemistry by Automated Quantitative Analysis, Arch. Pathol. Lab. Med., Vol 133, September 2009.

59. McLeod, Howard L.; James Cassidy, Robert H. Powrie, David G. Priest, Mark A. Zorbas, Timothy W. Synold, Stephen Shibata, Darcy Spicer, Donald Bissett, Yazdi K. Pithavala, Mary A. Collier, Linda J. Paradiso, John D. Roberts (July-2000). “Pharmacokinetic and Pharmacodynamic Evaluation of the Glycinamide Ribonucleotide Formyltransferase Inhibitor AG2034”. Clinical Cancer Research (American Association for Cancer Research) 6 (7): 2677-2684. PMID 10914709. http://clincancerres.aacrjournals.org/cgi/content/abstract/6/7/2677. Retrieved 2, Dec. 2008.

60. Avendano, Carmen; Menendez, J. Carlos (16, Apr. 2008). Medicinal Chemistry of Anticancer Drugs. Amsterdam: Elsevier. p. 37. ISBN 0-4445-2824-5.

61. Manegold C. Pemetrexed (Alimta, MTA, multitargeted antifolate, LY231514) for malignant pleural mesothelioma. Semin Oncol. 2003 August; 30(4 Suppl 10):32-6. PMID: 12917819

62. Raymond E, Faivre S, Armand J (2000). “Epidermal growth factor receptor tyrosine kinase as a target for anticancer therapy”. Drugs 60 Suppl 1: 15-23; discussion 41-2. PMID 11129168.

63. Jones H, Goddard L, Gee J, Hiscox S, Rubini M, Barrow D, Knowlden C, Williams S, Wakeling A, Nicholson R (2004). “Insulin-like growth factor-I receptor signalling and acquired resistance to gefitinib (ZD1839: Iressa) in human breast and prostate cancer cells”. Endocr Relat Cancer 11 (4): 793-814. doi:10.1677/erc.1.00799. PMID 15613453.

64. Blum G, Gazit A, Levitzki A (2000). “Substrate competitive inhibitors of IGF-1 receptor kinase”. Biochemistry 39 (51): 15705-12. doi:10.1021/bi001516y. PMID 11123895.

65. Warshamana-Greene G, Litz J, Buchdunger E, García-Echeverría C, Hofmann F, Krystal G (2005). “The insulin-like growth factor-I receptor kinase inhibitor, NVP-ADW742, sensitizes small cell lung cancer cell lines to the effects of chemotherapy”. Clin Cancer Res 11 (4): 1563-71. doi:10.1158/1078-0432.CCR-04-1544. PMID 15746061.

66. Los M, Roodhart J M, Voest E E (April 2007). “Target practice: lessons from phase III trials with bevacizumab and vatalanib in the treatment of advanced colorectal cancer”. The Oncologist 12 (4): 443-50. doi:1.1634/theoncologist.12-4-443. PMID 17470687.

What is claimed is:

1. A method for determining the histotype of a non-small cell lung cancer tumor which comprises:
(a) determining the levels of expression of each of thyroid transcription factor-1, cytokeratin-5, cytokeratin-13 and epidermal growth factor receptor in a sample from the non-small cell lung cancer tumor;
(b) calculating a score based on the levels of expression determined in step (a); and
(c) comparing the score obtained in step (b) with a predetermined reference score associated with histotypes of non-small cell lung cancer;
wherein the tumor is determined to be an adenocarcinoma if the score obtained in (b) is greater than the predetermined reference score and wherein the tumor is determined to be a squamous cell carcinoma if the score obtained in (b) is less than the predetermined reference score.

2. The method of claim 1, wherein the levels of expression of thyroid transcription factor-1, cytokeratin-5, cytokeratin-13 and epidermal growth factor receptor are determined using an automated pathology system.

3. The method of claim 1, wherein the levels of expression of thyroid transcription factor-1, cytokeratin-5, cytokeratin-13 and epidermal growth factor receptor are determined using a quantitative image analysis procedure.

4. The method of claim 1, wherein the sample is a tissue sample.

5. The method of claim 1, wherein the sample is a cytology specimen.

6. A method for determining whether a non-small cell lung cancer tumor is an adenocarcinoma which comprises:
(a) determining the levels of expression of each of thyroid transcription factor-1, cytokeratin-5, cytokeratin-13 and epidermal growth factor receptor in a sample from the non-small cell lung cancer tumor;
(b) calculating a score based on the levels of expression determined in step (a); and
(c) comparing the score obtained in step (b) with a predetermined reference score associated with histotypes of non-small cell lung cancer;
wherein the tumor is determined to be an adenocarcinoma if the score obtained in (b) is greater than the predetermined reference score.

7. The method of claim 6, wherein the levels of expression of thyroid transcription factor-1, cytokeratin-5, cytokeratin-13 and epidermal growth factor receptor are determined using an automated pathology system.

8. The method of claim 6, wherein the levels of expression of thyroid transcription factor-1, cytokeratin-5, cytokeratin-13 and epidermal growth factor receptor are determined using a quantitative image analysis procedure.

9. The method of claim 6, wherein the sample is a tissue sample.

10. The method of claim 6, wherein the sample is a cytology specimen.

11. A method for determining whether a non-small cell lung cancer is a squamous cell carcinoma which comprises:
(a) determining the levels of expression of each of thyroid transcription factor-1, cytokeratin-5, cytokeratin-13 and epidermal growth factor receptor in a sample from the non-small cell lung cancer tumor;
(b) calculating a score based on the levels of expression determined in step (a); and
(c) comparing the score obtained in step (b) with a predetermined reference score associated with histotypes of non-small cell lung cancer;
wherein the tumor is determined to be an squamous cell carcinoma if the score obtained in (b) is less than the predetermined reference score.

12. The method of claim 11, wherein the levels of expression of thyroid transcription factor-1, cytokeratin-5, cytokeratin-13 and epidermal growth factor receptor are determined using an automated pathology system.

13. The method of claim 11, wherein the levels of expression of thyroid transcription factor-1, cytokeratin-5, cytokeratin-13 and epidermal growth factor receptor are determined using a quantitative image analysis procedure.

14. The method of claim 11, wherein the sample is a tissue sample.

15. The method of claim 11, wherein the sample is a cytology specimen.

16. A method for selecting an appropriate therapeutic treatment or combination of therapeutic treatments for a patient afflicted with non-small cell lung cancer comprising:
(a) determining the levels of expression of each of thyroid transcription factor-1, cytokeratin-5, cytokeratin-13 and epidermal growth factor receptor in a sample of the non-small cell lung cancer tumor from the patient;
(b) calculating a score based on the levels of expression determined in step (a); and
(c) comparing the score obtained in step (b) with a predetermined reference score associated with histotypes of non-small cell lung cancer;
(d) assigning a histotype to the non-small cell lung cancer wherein the tumor is determined to be an adenocarcinoma if the score obtained in (b) is greater than the predetermined reference score and wherein the tumor is determined to be a squamous cell carcinoma if the score obtained in (b) is less than the predetermined reference score; and
(e) selecting a therapeutic treatment based on the histotype assigned in step (d).

17. The method of claim 16, wherein the therapeutic treatments are selected from the group consisting of erlotinib, gefitinib, bevacizumab, sorafenib, docetaxel, gemcitabine, pemetrexed, and cisplatin.

18. The method of claim 16, wherein the levels of expression of thyroid transcription factor-1, cytokeratin-5, cytokeratin-13 and epidermal growth factor receptor are determined using an automated pathology system.

19. The method of claim 16, wherein the levels of expression of thyroid transcription factor-1, cytokeratin-5, cytokeratin-13 and epidermal growth factor receptor are determined using a quantitative image analysis procedure.

20. The method of claim 16, wherein the sample is a tissue sample.

21. The method of claim 16, wherein the sample is a cytology specimen.
